(19) United States
(12) Patent Application Publication
(10) Pub. No.: US 2021/0358619 A1
(43) Pub. Date: Nov. 18, 2021

(54) Health Sticker: A Modular Adhesive Platform Monitoring Vital Signals

(71) Applicant: The Regents of the University of Colorado, a body corporate, Denver, CO (US)

(72) Inventors: Tam Vu, Boulder, CO (US); Robin Deterding, Boulder, CO (US); Nam Bui, Broomfield, CO (US); Nhat Pham, Boulder, CO (US)

(21) Appl. No.: 17/390,529

(22) Filed: Jul. 30, 2021

Related U.S. Application Data
(63) Continuation-in-part of application No. PCT/US2020/015961, filed on Jan. 30, 2020.
(60) Provisional application No. 62/798,965, filed on Jan. 30, 2019.

(51) Int. Cl. G16H 40/67; A61B 5/00; G16H 10/20; G16H 50/30; G16H 50/20; G16H 10/60; G16H 50/70; G16H 15/00; G16H 20/10; G06F 1/28

(52) U.S. Cl. CPC G16H 40/67; A61B 2560/0214; A61B 5/7405; A61B 5/748; A61B 5/7264; A61B 5/746; A61B 5/0022; A61B 5/4842; A61B 5/4833; G16H 10/20; G16H 50/30; G16H 50/20; G16H 10/60; G16H 50/70; G16H 15/00; G16H 20/10; G06F 1/28; A61B 2560/0431; A61B 2560/045; A61B 5/742

(57) ABSTRACT

Systems and methods for medical monitoring devices are provided. Various embodiments include a monitoring device platform. The platform may adhere to a smartphone and house one or more medical monitoring devices to accommodate for one or more health conditions. The medical monitoring devices may be interchangeable and the types of medical monitoring devices on the platform is customizable. The platform may include Bluetooth® functionality and is compatible with smart phones or other smart devices. Medical information collected by the platform is transmitted to the smart phone for viewing by the user. The medical information may be processed by a machine learning engine and can be used to highlight relevant information within an action plan created by a medical professional. Individual medical devices can slide and lock into the platform and may be used while the phone is in use.

### CROSS-REFERENCE TO RELATED APPLICATIONS

This application is a continuation-in-part of International Application No. PCT/US2020/015961 filed Jan. 20, 2020, which claims priority to U.S. Provisional Application Ser. No. 62/798,965 filed Jan. 30, 2019, both of which are incorporated herein by reference in their entireties.

### TECHNICAL FIELD

Various embodiments of the present technology generally relate to medical devices. More specifically, the embodiments of the present technology relate to modular adhesive devices for monitoring vital signals.

### BACKGROUND

The need for integrated and personalized medical devices is ever growing. Currently, medical devices monitor various bodily functions of a user often exist as dedicated separate devices that each only take a fixed number (e.g., one or two) of readings. For example, a diabetic person may carry a glucose meter configured to only monitor blood sugar levels. However, if this person has additional medical monitoring needs, they will be forced to carry multiple and separate devices. This can make providing broad spectrum and holistic medical coverage difficult from a doctor's perspective and tedious from the view of the patient. Additionally, many existing devices often fail to present medical information in a complete and easily digestible manner to non-medical professionals. The screens and user interfaces of these devices pale in comparison to that of the modern smartphone further compounding the problem of monitoring the health of an individual.

Additionally, the ability of the personal user devices to track changes in vital signals or other bodily functions of a patient or user are limited. Information obtained from medical monitoring devices is often either recording in a rudimentary way or must be tracked manually by writing down information obtained from the medical device. This can make it difficult for a medical professional to monitor the health status of a patient when the patient is not at a hospital or office setting. Similarly, if a person has multiple vital signs that need monitoring, information regarding these vital signs is distributed between multiple medical devices, and therefore difficult to collate into single information source further compounding the difficulties medical professionals face.

As such, there are a number of challenges and inefficiencies created in traditional medical monitoring devices. For example, traditional monitoring devices are unable to integrate with the smartphone of a user. Thus, it can be difficult to provide personalized and on-demand monitoring of bodily functions. It is with respect to these and other problems that embodiments of the present invention have been made.

### SUMMARY

Systems and methods are described for a health sticker platform to monitor various vital signals and/or other biological measures of states of a user. The health sticker may take the form of a modular medical device attaching to the back of a mobile device (e.g., a cell phone or smartphone) to capture the vital signals of a user. The health sticker can support a variety of modules to accommodate different health conditions. These modules may be interchangeable and customizable depending on the specific medical condition or monitoring needs of a user. For instance, a health sticker may include a module for monitoring the heart rate of a user, as well as a module for monitoring the body temperature of the user and any other modules needed to assess a medical condition of the user. It should be appreciated that the number of modules on the platform, nor the type of modules on the platform are limited. For instance, certain modules can be invasive to measure concentrations of blood components or other bodily functions that require an invasive approach while another module can be non-invasive. The health sticker may further include a battery or other power interface to supply power to modules on the platform. The health sticker can include various communication functionality (e.g., Bluetooth) to connect the health sticker to a mobile device or other type of computing device. Alternatively, the health sticker may plug into a mobile device or other type of computing device and operate via a wired connection.

In some embodiments, a system to facilitate medical diagnostics is presented. The system can be a medical device platform (e.g., a health sticker) that includes an adhesive side and a device side. The adhesive side of the platform can include an adhesive layer which can bind the medical device platform to a mobile device (e.g., a smartphone). The adhesive layer can take the form of a glue, a tape, or other types of adhesive material. In other embodiments, mechanical features (with or without an adhesive layer) may be used to secure the platform to the mobile device. Such mechanical features may include, for example and without limitation, swing out or pop up mechanisms.

The device side can include one or more device ports (e.g., 1-10 ports) which hold one or interchangeable modular medical devices. In accordance with various embodiments, each of the device ports may include a slide and lock mechanism which holds in place a modular medical device inserted into the device port. The device ports may also include a device adapter to connect the one or more medical devices to the platform. The device adapter may be a USBC adapter or other type of adapter capable transferring analog or digital information. The medical devices can be invasive or non-invasive and can be used to measure any type vital sign or biometric signal or state of the user. In some embodiments, the one or more medical devices can collect the one or more vital signs of the user as medical information.

In various embodiments, the system can include a data module to aggregate the medical information collected by the medical devices stored in the device ports of the platform. For example, in some embodiments, the module can be configured to develop correlations between medical information originating from distinct modular medical devices on the platform to determine medical information that is not directly collected by any of the one or more modular medical devices. A communication module can be included to transfer the medical information from the platform to the mobile device or other type of computing device. Alternatively, the communication module may transfer the medical information to some other type of device such as a laptop computer. In some embodiments, the system can further include a power management module and a rechargeable battery. The battery can supply power to the components of the medical device platform and any medical devices connected to the platform. In some embodiments, the power management module can detect when a medical device is inserted or removed from a device port and then notify the user of the availability or removal of the interchangeable medical device.

Some embodiments may cause a graphical user interface (GUI) to be generated for the presentation of medical information collected via the interchangeable medical devices and/or from various data stores (e.g., electronic medical records, etc.). The GUI may present one or more modes of operation for presenting medical information, informing the user about the medical information, and/or controlling the operating of modular medical devices stored on the platform. In some embodiments, the operating mode of the GUI can present the medical information as more or more visual or textual elements. These visual and/or textual elements can be plots, charts, tables, numbers, symbols, or words, or any other element which aids in the understanding of the medical information. The operating mode may update, in real time, any of the visual and/or textual elements in response to changes in the medical information. For example, if the operating mode depicts the breathing rate of the user, the operating mode can update the one or more visual or textual elements in response to a change in the breathing rate of the user.

In other embodiments, the GUI can include a mode of operating that includes visual and/or audio notifications which trigger in response to a change in the medical information. For example, a sudden change in the body temperature of the user can cause the triggering of a notification. The audio and/or visual notifications may include pings, flashes, or tooltips, or any other type of audio or visual notification. Other operating modes of the GUI can include a variety of switches or other mechanisms to control each of the one or more medical devices. The switches may correspond to a specific device port and can activate or deactivate the medical devices. Additionally, the switches may change the operating modes of the medical devices. For example, a single medical device may have a variety of operating modes, and the management module can include switches to toggle between operating modes of the medical device.

In some embodiments, the GUI of the system may incorporate an interactive questionnaire which presents questions to the user. The questions can be dynamically generated (e.g., based on current environmental conditions, medical readings, medical history, and/or other factors) and may relate to a current medical condition of the user or to the general health of the person. Completion of the interactive questionnaire by the use may determine a severity level of the medical condition of the user. The severity level or health severity zone may be represented as a health score or other type of alpha/numeric representation.

In some embodiments, the system can include an artificial intelligence or machine leaning engine which processes the medical information of the user to further determine the severity level of the medical condition. The system may combine the severity level determined by the interactive questionnaire with the severity level determined by the machine learning engine to create an aggregate health score. The aggregate health score may form the basis of an action plan (or indexing into an existing action plan) for treating the medical condition of the user. The action plan can include one or more prescribed responses to treat or otherwise address the current state of the medical condition of the user. Additionally, the system can include a communications module to transmit health information to an external health resource. The information may include the answers from the interactive questionnaire, the aggregate health score, or any other information relevant to the health of the user. The user may be able to provide answers to the questionnaire and/or ask additional questions via a video or chat interface. In some embodiments, the external health resource may be a device operated by hospital or medical professional.

Embodiments of the present technology also include computer-readable storage media containing sets of instructions to cause one or more processors to perform the methods, variations of the methods, and other operations described herein.

Various embodiments can include methods to facilitate medical diagnostics. In some embodiments, various medical states (e.g., vital signals, etc.) can be measured through the use of a modular medical device platform. The recorded measurements can then be correlated (e.g., though the use of machine learning) to a medical condition of the user. Correlating the recorded measurements (e.g., of vital signs, blood glucose level, pulmonary function, etc.) to the medical condition can include developing a health score indicative of the current state of the medical condition of the user. In some embodiments, portions of an existing action plan created by a medical professional (e.g., doctor) can be highlighted for execution by the user. In some embodiments, the action plan can be created, modified, and/or indexed into based on the health score or zone of the user. The activities within the action plan can then be presented to user. The action plan may in some instances include a prescribed response to the current state of the medical condition. Once the action plan is presented to the user, various techniques may be used to confirm that the user has followed the action plan (e.g., through the use of push notifications, text notifications, or the like).

In some embodiments a health questionnaire can be presented to the user. The questionnaire may be presented via a GUI or other similar interface and include questions relevant to the current state of the medical condition of the user. Upon completion of the health questionnaire, the current state of the medical condition can be determined. Furthermore, the answers to the health questionnaire may be used to create, modify, and/or index into the action plan.

Some embodiments can include a cloud-based data aggregation engine to facilitate medical diagnostics. The cloud-based data aggregation engine may include a communications module to receive medical information from one or more mobile devices. The mobile devices may be equipped with a modular medical device platform configured to measure the vital signs of a user and develop medical information of the user based on the vital signs. The mobile device may be further configured to report the medical information to the cloud-based data aggregation engine. In other embodiments, the cloud based-data aggregation engine can include a machine learning engine to ingest the medical information and process the medical information to develop a health score of each user. The health score can indicate the current state of a medical condition of each user. The cloud-based data aggregation engine may also include a health assessment module configured to distributed health questionnaires to the users. Answers to the health questionnaire can be used to update the health scores generated by the machine learning engine. In other embodiments, the machine learning engine may generate predictions to determine a future state in the medical condition of each user based on the ingested medical information. The predictions may include determining if a patient's medical condition is improving or deteriorating.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the scope of the present invention. Accordingly, the drawings and detailed description are to be regarded as illustrative in nature and not restrictive.

### BRIEF DESCRIPTION OF THE DRAWINGS

Embodiments of the present technology will be described and explained through the use of the accompanying drawings.

FIG. 1 illustrates an exemplary operating environment of a cloud-based diagnostics system according to one or more embodiments of the present technology.

FIGS. 2A-2B illustrate a health sticker platform in accordance with some embodiments of the present technology.

FIGS. 3A-3B illustrate a health sticker platform according to one or more embodiments of the present technology.

FIG. 4 illustrates a module and module platform according to some embodiments of the present technology.

FIG. 5 is block diagram depicting a health sticker in accordance with some embodiments of the present technology.

FIG. 6 is a block diagram illustrating examples of a health monitoring system according to one or more embodiments of the present technology.

FIGS. 7A-7C illustrates a modular medical monitoring platform according to one or more embodiments of the present technology.

FIG. 8 is a flowchart for operating an integrated health monitoring system according to one or more embodiments of the present technology.

FIG. 9 is a sequence diagram depicting the operating of an integrated health monitoring system in accordance with some embodiments of the present technology.

FIG. 10 is a sequence diagram depicting the operating of an integrated health monitoring system in accordance with some embodiments of the present technology.

FIGS. 11A-11B illustrate a modular medical monitoring platform according to one or more embodiments of the present technology.

FIGS. 12A-12B illustrate a health sticker and medical monitoring modules according to one or more embodiments of the present technology.

FIG. 13 is an example of printed circuit board of a health sticker in accordance with one or more embodiments of the present technology.

FIG. 14 illustrates a medical monitoring module according to one or more embodiments of the present technology.

FIG. 15 is a plot illustrating the results of a predictive machine learning engine that may be used in one or more embodiments.

FIGS. 16A-16D are plots illustrating the results of a predictive machine learning engine according to one or more embodiments of the present technology.

FIGS. 17A-17D illustrate a health sticker platform in accordance with some embodiments of the present technology.

FIG. 18 is a data structure providing a template for programming algorithms to link monitoring input to care coordination treatment plans to facilitate personalized provider driven patent treatment plans, according to some embodiments of the present technology.

FIG. 19 is a block diagram depicting an exemplary computing apparatus according to one or more embodiments of the present technology.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Various embodiments of the present technology relate generally to a portable and modular medical platform for monitoring patients. Currently, there is no efficient way for medical professionals to continuously and effectively monitor the health of a patient when the patient is not at the hospital. Current medical monitoring equipment is often too large and expensive for home use. Additionally, this equipment is not easily mobile and/or integrated into mobile and online communications systems. Problems are further compounded when a patient needs to be on medication. Medical professionals lack the ability to automatically prompt patients when to take medication, instruct patients on the type of medication needed, determine the amount of medication needed at a particular moment, or determine if a patient has taken the medication prescribed to them.

In addition to medical grade solution, there are some end-user solutions that measure only vital signs (e.g., heart rate). However, these traditional end-user devices that can measure vital signs are fixed system (e.g., they are not expanded modular systems) and only take a fixed set of measurements. Moreover, these end-user devices do not integrate a holistic health platforms capable of identifying symptoms and environmental condition. In addition, these traditional end-user devices do not have a feedback look for education, warnings and adherence to medically recommend treatment all built into one device. In contrast, various embodiments of the present technology can include a hardware platform that includes one or more interchangeable modular medical devices, software to operate the hardware platform that connects patients with medical professionals, and an artificial intelligence and/or machine learning engine to interpret medical data collected by the medical device. Due to the difficulty in combining software with miniature and modularized medical devices, medical platforms similar to the one described herein have yet to be developed. In short, the present technology provides a holistic and closed loop solution for patients and medical professionals.

Some embodiments provide for a mobile hardware platform that can be connected to a mobile device. The mobile hardware platform may be configured to adheres to the reverse side of a mobile device or mobile device case. The hardware can include a number of device ports each configured to securely house an interchangeable modular medical monitoring device. The mobile hardware platform may operate with a wide range of interchangeable modular medical monitoring devices. For example, one device port can house a modular medical device to monitor SpO₂ levels and heart rate while a different device port can house a modular medical device configured to monitor blood glucose levels. Depending on the patient's medical condition, the medical monitoring devices housed in the hardware platform may vary. The device ports can include a docking mechanism (e.g., a USBC port) to allow for transmission of collected medical information, modular medical device identifiers, and/or other data from the modular medical devices. The device ports may also include a locking mechanism (e.g., clamps, press fitting, spring loaded arms, etc.) to hold the modular medical devices in place within the ports. The modular medical devices may be designed to be small, allowing for two or more to fit comfortably behind the mobile device. In accordance with various embodiments, the modular medical devices can be inserted into any device port and that the device ports may be non-specific. For example, a single device port is not limited by the type of medical device.

When in use, the modular medical device can transmit a unique device ID to notify the smartphone that the medical device is being used. The medical device can extend or unfold from the platform thus allowing the user to measure a vital sign while viewing the screen of the mobile device. When the medical device is being used by the patient, the hardware platform can collect the medical information and transmits the medical information to the phone (e.g., via Bluetooth) where the medical information can be presented to the user. The platform can include an on-board battery to provide power to the attached medical devices. In other embodiments, the platform may secure power from the mobile device (e.g., via a USBC connector, a Lighting connector, via wireless power transfer, Near-Field Communication (NFC), or the like). In some embodiments, the platform may detect when a medical device is not in use and will stop providing power to that device as a means to conserve battery power.

Some embodiments can use a software application that can utilize information collected by the hardware platform and present the medical information to the user in an easily understandable manner. The software can collect the medical information in real time and generate plots, graphs, or other visual to help the user understand the medical data. Additionally, the software may relay the collected information to a medical professional so that the medical professional can assess the current status of the patient. In some situations, the medical professional, or the software itself, can generate an alert to notify the patient to take a variety of actions. The alert may direct the patient to go to the hospital or may direct the patient to take a specific amount and/or type of medication. However, the type of action dictated by the alert is not limited. The software can additionally include incentives to persuade the user to follow the notifications. The software may report if a patient acknowledged the notification to allow medical professionals to tell if a patient responded to the notification and confirmed that they followed the advised course of action. In some embodiments, the alerts may request that the user send a video to another person (e.g., parent or guardian or health care facility) and a chat function.

The software may be further tailored to implement an action plan for the patient to take. The action plan can be uploaded to the software by the medical professional and can provide the user with notifications or instructions about how, or when to treat a medical condition. In some embodiments, the action plan may be provided to the user by the medical professional. Then, the user can access an interface to upload or report what the action plan says. For example, the plan may be scanned, typed in, or entered via an interactive user interface. The system can then confirm the action plan came from a medical professional.

Alternatively, aspects of the action plan may be highlighted by the software in response to medical information. In some embodiments, the software can include a questionnaire, with questions relating to the current medical status of the patient and is filled out by the patient or a guardian responsible for the patients care. The results are then ingested by the software application and can then be transmitted to the medical professional to address the current medical condition of the patient. Alternatively, the software may have pre-programmed responses that align with medical literature to answers provided by the patient or the patient's medical guardian. For example, if the guardian provides a specific answer to a question, the software can automatically identify a response in the action plan based on the answer. Either way, the software helps to formulate a severity level relating to the patient's condition so that the patient and the medical professionals can take the appropriate course of action.

In some embodiments, the aggregate health score may correspond to levels or zones within an existing action plan provided by a medical professional. For example, a normal aggregate health score may correspond to a green or normal zone of an action plan while a severely abnormal health score may correspond to red zone or an action plan. The zones of the action plan can be associated with a plurality of actions given by a medical professional or indicated by the medical literature. For illustrative purposes, an example action plan is provided.

| | | | Vitals and/or | |
|---|---|---|---|---|
| Severity Level | Medications/Actions | Symptoms | Bio-signals | Environment |
| Green/Normal | Baseline/Regular | None | Normal | Good |
| Zone or Normal | Medication | | | |
| Health Score | | | | |
| Yellow/Moderate | Additional | Symptoms | Mildly | Abnormal |
| Zone or | Medication | Present | Abnormal | |
| Abnormal Health | Education Provided | Mild to | | |
| Score | Warning and | Moderate | | |
| | Notification | | | |
| | Re-evaluate | | | |
| Red/Severe | Additional | Symptoms | Moderate to | Dangerous for |
| Zone or Very | Medication | Present | Severely | Patient Health |
| Abnormal Health | Education Provided | Moderate to | Abnormal | |
| Score | Warning and | Severe | | |
| | Notification | | | |
| | Instructions to | | | |
| | Contact Health | | | |
| | Professional | | | |
| | Re-Evaluate | | | |
| | Assessment | | | |

In some embodiments, the software may exist as a distributed cloud-based system. Some elements of the software can exist in the cloud while other elements are stored locally on the mobile device of the user. The cloud-based system can be configured to receive medical information from a mobile device of the patient or guardian and identify the appropriate response in the action plan based on the medical information. The cloud-based system may then send the action with the appropriate response highlighted to the patient of to the guardian of the patient. The cloud-based system may further relay communications between a medical professional or other type of health resource and the mobile device. In some examples the cloud-based system will send one or more notifications to the mobile device of the patient or guardian to signify the presence of an action plan. The cloud-based system may record when or if the notification was acknowledged by the patient or guardian.

In various embodiments, the cloud-based system may detect abnormalities in the medical information received from the mobile device. When an abnormality is observed, the cloud-based system can report the abnormality to the mobile device and identify a portion of an action plan used to treat the abnormality. The cloud-based system may additionally track when and if a patient takes medication, analyze symptoms of a patient, and analyze the environmental conditions of a patient's location to identify portions of an action plan to address a medical condition of the patient. During the analysis of the medical information, the cloud-based can develop longitudinal health trends of a patient's medical condition. For example, the longitudinal health trends may include a patient's medical history. Once developed, the cloud-based system may relay the longitudinal trends to the mobile device of the patient. The cloud based-system may track patient's adherence to an action plan through the longitudinal trends.

In contrast, further embodiments include a machine learning engine that ingests, and processes medical information collected by the hardware platform. The machine learning engine may exist either locally on the user device, on the hardware platform, may be stored remotely, or may exist in a cloud-based system. Medical information collected from the medical devices on the hardware platform can include vital signs or other biological signals. The medical information may be examined by the engine to aid in the health assessment of the patient. In certain circumstances, conditions undetected by the medical professionals or the software may be uncovered by the machine learning engine. The machine learning engine can assign a score or severity level to the received medical information to help the medical professional understand the patient's current situation. The assessment by the machine learning engine can be combined with the information provided by the software to identify relevant portions of the action plan. Thus, a proper response can be established in real time while the patient is outside of the hospital to facilitate in the treatment of a wide variety of medical conditions.

Various embodiments of the present technology provide for a wide range of technical effects, advantages, and/or improvements to medical diagnostics systems. For example, various embodiments include one or more of the following technical effects, advantages, and/or improvements: 1) integrated platform and medical devices that provides a combination of hardware with interchangeable modular sensors, software platforms integrated data from the integrated modular sensors and capable of identifying symptoms and environmental condition, and machine learning; 2) devices providing feedback loops for education, warnings, and adherence to medically recommend treatment all built into one device; 3) design of a device having interchangeable modular sensor to reduce the number of independent medical monitoring devices needed by a patient; 4) integrating medical monitoring devices with mobile devices; 5) modular design providing a portable and customizable medical monitoring platform; 6) hardware and software to use non-routine computer operations, components, and designs to organize disparate and independent medical information; 7) a user interface to present information on a wide variety of vital signals in an easily understandable manner; 8) use of machine learning and non-routine computer operations to tabulate medical information thereby creating a personalized medical history of various vital signs of a user; 9) easily interchanging medical monitoring devices on a single portable platform; 10) health platform and devices that provide immediate and easy to follow health action plan prescribed by health professional as home treatment or recommendations based upon the medical literature directly to the user via the smart phone; and/or 11) integrated platform to provide video to a guardian or health system with a chat function.

In the following description, for the purposes of explanation, numerous specific details are set forth in order to provide a thorough understanding of embodiments of the present technology. It will be apparent, however, to one skilled in the art that embodiments of the present technology may be practiced without some of these specific details. While, for convenience, embodiments of the present technology are described with reference to a medical monitoring platform that can adhere to the back of a smart phone, where the platform can accommodate one or more medical monitoring devices and utilize the smart phone to present medical information obtained from the medical monitoring devices in an easily understandable manner.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

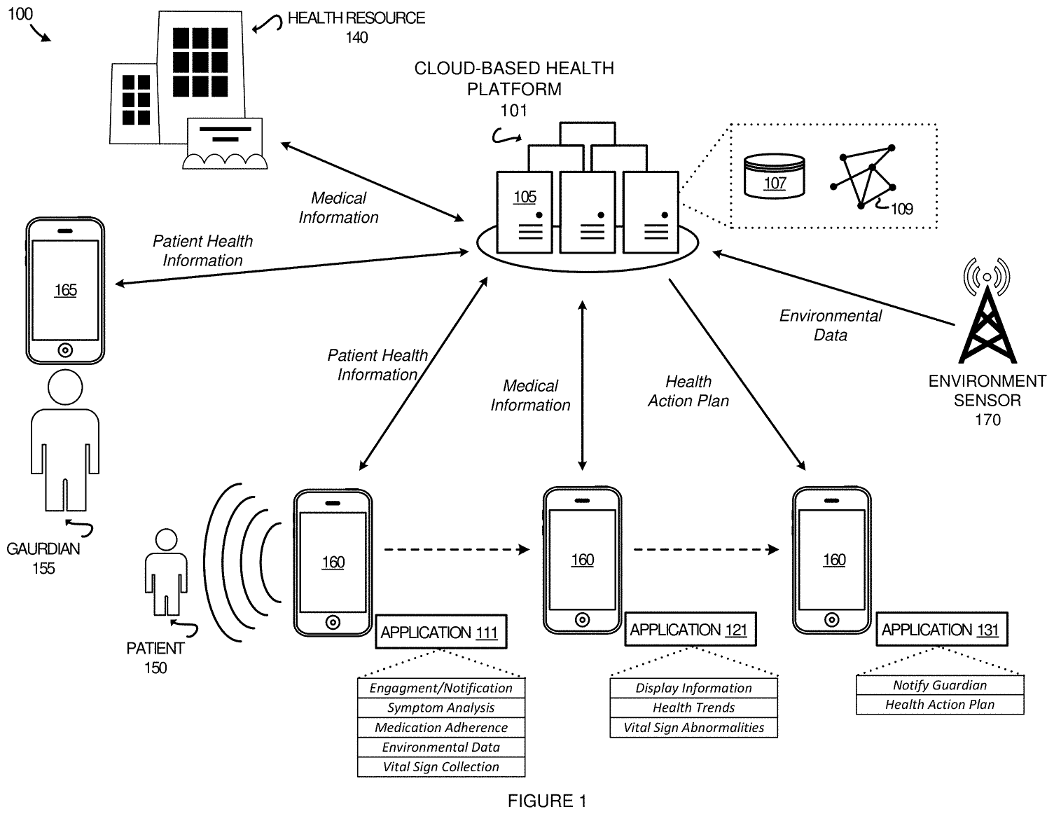

FIG. 1 illustrates an example of operating environment 100 in which some embodiments of the present technology may be utilized. As illustrated in FIG. 1, operating environment 100 may include cloud-based health platform 101, health resource 140, patient 150, guardian 155, patient mobile device 160, guardian mobile device 165, environment sensor 170, and applications 111, 121, and 131 which can operate on mobile device 160. Cloud-based health platform 101 can include data center 105. Data center 105 may exist as one or more computing devices integrated into a network that communicates with mobile device 160. Examples include, but are not limited to, server computers and data storage devices deployed on-premises, in the cloud, in a hybrid cloud, or elsewhere. Data center 105 is communicatively coupled to mobile device 160 and health resource 140. Data center 105 may relay communications between health resource 140 and mobile device 160. In some embodiments, data center 105 can include diagnostics system 107 and machine learning engine 109. Diagnostics system 107 and machine learning engine 109 may be implemented as one or more software programs, applications, data networks, neural networks, or other types of software or hardware configured to process medical information. Diagnostics system 107 and machine learning engine 109 may exist a single unit or may be distinct.

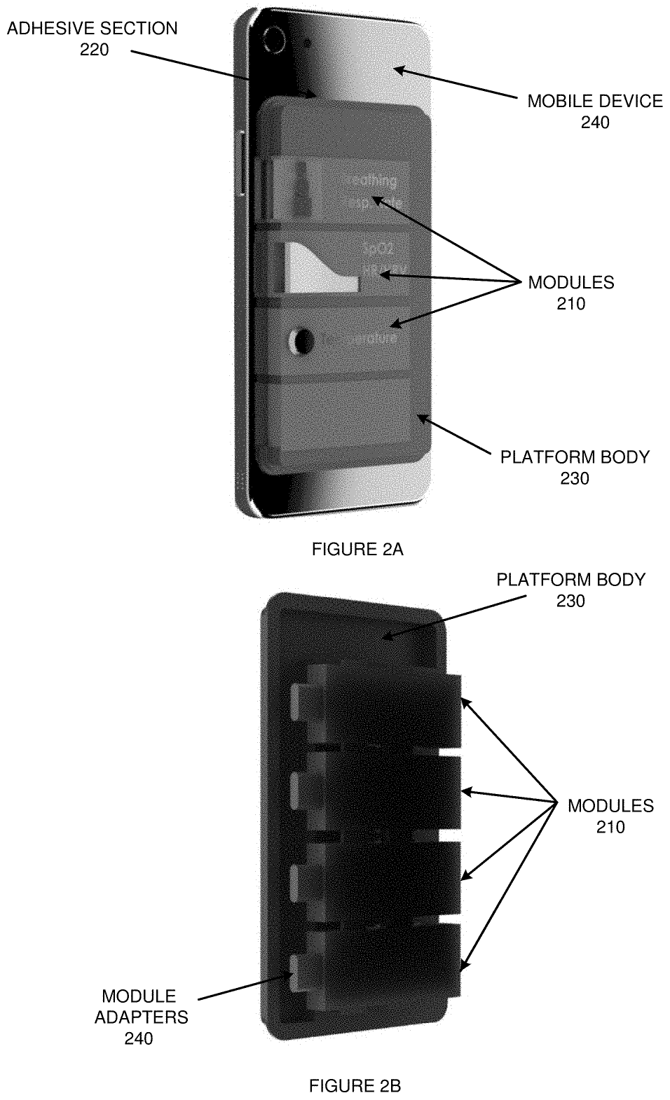

Mobile device 160 is representative of one or mobile devices which examples including, but not limited to, cell phones, smartphones, tablet computers, smart watches, or other types of mobile devices. Mobile device 160 may be configured to measure the vital signs of patient 150. Examples of vital signs of patient 150 may include the breathing rate, peripheral capillary oxygen saturation (SpO₂) levels, heart rate, respiratory or other types of biological information such as blood glucose levels, weight, movement or pulmonary function. In some embodiments, mobile device 160 is equipped with external medical sensors (shown in FIGS. 2A-2B and others) for measuring the vital signs. The medical sensors may be integrated into a case of mobile device 160 or may adhered as a sticker to the external body of mobile device 160. Alternatively, mobile device 160 may include the medical sensors as native hardware. It should be noted that the types of medical sensors included with mobile device 160 are not limited nor is the mode in which the medical sensor exist on mobile device 160.

In some embodiments, mobile device 160 measures one or more vital signs of patient 150. When measuring the vital signs of patient 150, mobile device 160 may run application 111 to facilitate the health assessment of patient 150. Application 111 operating on mobile device 160 can be configured to receive a health questionnaire from cloud-based health platform 101. The health questionnaire may include questions relating to the medical condition of patient 150, questions relating to the medication adherence of patient 150, questions pertaining to the type of prescribed medication of patient 150, questions about when or if the vital signs of patient 150 were measured, and questions relating to the symptoms of patient 150. In further embodiments, application 111 may receive environmental data relating to the medical condition of the patient. Examples of environmental data include, but are not limited to, air pollen levels, air pollution levels, UV levels, or locations where disease is prevalent, or other environmental factors that can affect the health of patient 150. Application 111 may include educational information, warnings, or recommendations regarding the environmental data and can present the educational information, warnings or recommendations to patient 150 and/or guardian 155. In some embodiments, environmental data is collected by environment sensor 170. Upon receiving the health questionnaire, application 111 then presents the health questionnaire and any other information relating to the health of patient 150, to patient 150 via mobile device 160. Application 111 then records the responses to the questionnaire by patient 150 as medical information.

In some embodiments, if patient 150 is unable to operate mobile device 150, diagnostic system 101 may instead deliver the health questionnaire, and any relevant information regarding the health of patient 150, to guardian mobile device 165. Examples of when patient 150 cannot operate mobile device 160 may occur if patient 150 is a young child or if patient 150 is geriatric. If this is the case, information regarding patient 150 can be sent to guardian mobile device 165 where the information is assessed by guardian 155.

Application 111 may inform either patient 150 or guardian 155 to measure one or more vital signs of patient 150 through the use of medical monitoring devices adhered or embedded into mobile device 160. Application 111 may present one or more mechanisms on mobile device 160 to allow patient 150 or guardian 155 to control, activate, or deactivate any medical devices operatively coupled to mobile device 160. Application 111 may further collect the measured vital signs or other biological signs of patient 150 as medical information.

In various embodiments, cloud-based health platform 101 may instruct application 111 to engage patient 150 or guardian 155. Application 111 can generate or more notifications and display the notifications on mobile device 160. In some examples, application 111 may generate a text message to engage patient 150 or guardian 155. The notifications may include instructions sent by health resource 140 to take a variety of actions regarding the health of patient 150. For example, the instructions may include directions to take or administer a specific amount or type of medication, directions on when to take or administer the specific amount or type or medication, instructions to begin monitoring one or more vital signs of patient 150, instructions to recheck the vital signs of patient 150, instructions that share medical advice given by a health professional, or instructions to go a hospital. Application 111 may generate additional notifications if either patient 150 or guardian 155 fail to acknowledge the instructions. In some embodiments, application 111 may include a reward system to incentive guardian 155 or patient 150 to acknowledge the notification generated by application 111 or follow instructions sent by health resource 140. Application 111 may further include an education system that delivers general education regarding the medical condition of patient 150 to patient 150 and/or guardian 155.

Once application 111 has finished collecting medical information from user 111, application 121 may initiate and send the medical information to cloud-based health platform 101 or to elements within cloud-based health platform 101. The medical information may include the symptoms of patient 150, vital sign measurements of patient 150, the medication adherence of patient 150, or environmental data that may affect patient 150. Application 121 may display one or more graphics on mobile device 160 that illustrate the measured vital signs of patient 150 or any other medical information. The graphic presentation of medical information may occur in real time and update in response to changes in the measured vital signs. Application 121 may additionally generate audio or visual alerts or signals in response to a change in the medical information or when an anomaly in the medical information is detected. In some embodiments, application 121 may detect an abnormality in the vital signs of patient 150. Application 121 can cross-reference the measured vital signs of patient 150 with medical literature to determine if the vital signs of patient 150 fall within an abnormal range. If application 121 determines the measured vital signs to fall in an abnormal range, application 121 can then notify or request an action plan from cloud-based health platform 101. Application 121 may additionally generate a warning or notification for patient 150 and/or guardian 155 that the measured vital signs fall within an abnormal range.

After receiving the medical information, cloud-based health platform 101 may relay the medical information to health resource 140, diagnostics system 107, and machine learning engine 109. Diagnostics system 107 processes and stores medical information relating to patient 150. In some embodiments, diagnostics system 107 can include one or more action plans created by a medical professional associated with patient 150 that relate to the health condition of patient 150. Each action plan can include a prescribed response or treatment option to address the current state of the medical condition of patient 150. In some embodiments, diagnostics system 107 can assign a score to the medical information of patient 150 to assist in identifying the relevant portions of an action plan for responding to a current medical condition of patient 150.

In other embodiments, machine learning engine 109 may ingest the medical information and process the medical information to generate a prediction, based on the ingested medical information, of the current state of the medical condition of patient 150. Machine learning engine 109 may represent the prediction as a health score. In some embodiments, machine learning engine 109 may ingest the health score generated by diagnostics system 107 and use this health score to update the prediction and then generate an updated health score based at least in part on the updated prediction.

Machine learning engine 109 may also provide predictions relating to a future medical condition of patient 150. For example, machine learning engine 109 may generate a personalized warning tailored for patient 150 after ingesting the medical information of patient 150. After generating the updated health score, machine learning engine 109 can transfer the updated health score to diagnostics system and diagnostics system can utilize the updated health score when identifying the relevant sections of an action plan. In some examples, diagnostics system 107 may populate an action plan with the medical information obtained from patient 150 and predictions generated by machine learning engine 109. When populated with this information, the action plan can be more easily understood by both patient 150 and guardian 155.

Once the relevant portions of an action plan have been identified by diagnostics system 107, cloud-based health platform 101 notifies and then transmits the action plan to mobile device 160. When the action plan is received, application 131 initiates and displays the action plan on mobile device 160. In some embodiments, application 131 generates one or more audio or visual notifications to inform patient 150 about the action plan. Application 131 may additionally request confirmation from patient 150 or guardian 155 that patient 150 followed the prescribed response of the action plan. In should be appreciated that applications 111, 121, and 131 can be separate and distinct applications or can exist as different operating modes of a single unified application.

FIG. 2A depicts a modular health platform and phone case according to some embodiments of the present technology. FIG. 2B shows an alternative viewing angle of the modular health platform and the phone case as shown in FIG. 2A. In some embodiments, the modular health platform may include modules 210, adhesive section 220, and platform body 230. Modules 210 can include one or more sensors for measuring the vital signs or other biometric signals of a user. Adhesive section 220 may cover one side of platform body 230 and affix platform body 230 to mobile device 240. Platform body 230 may include one or more slots health platform configured to hold modules 210. The one or more slots can exist as recessed sections on platform body 230. The recessed sections can be shaped in a way to accommodate the shape of any one of modules 210. Modules 210 that are inserted into platform body 230 can removed from the health platform and are interchangeable. For instance, a module of modules 210 for measuring the breathing rate of a user may be removed from the platform body 230 and then replaced by a different module of modules 210 that measures the heart rate of a user.

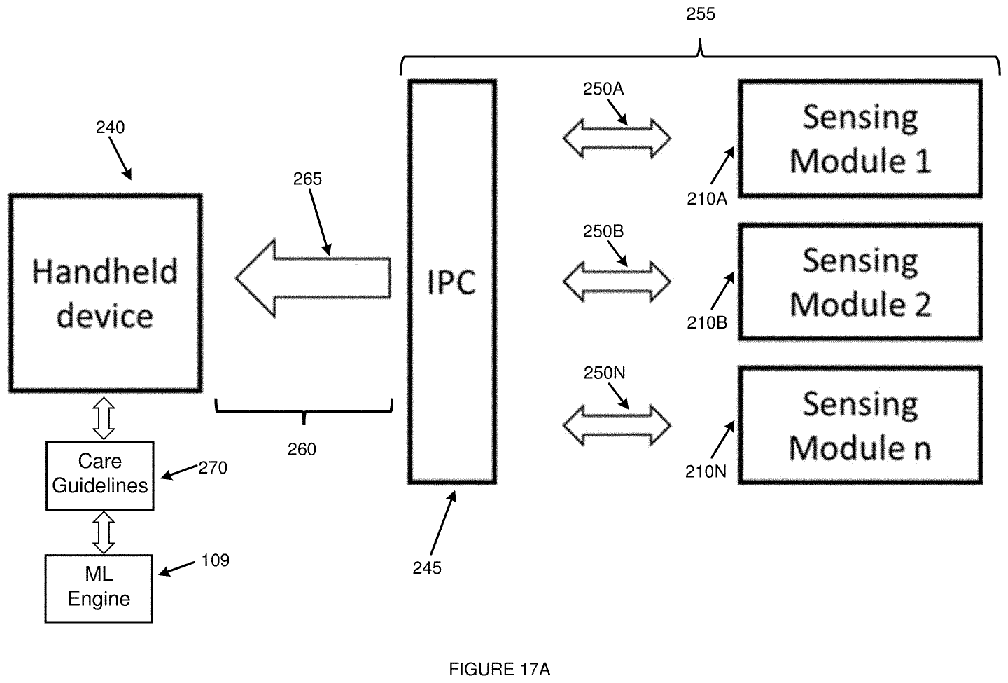

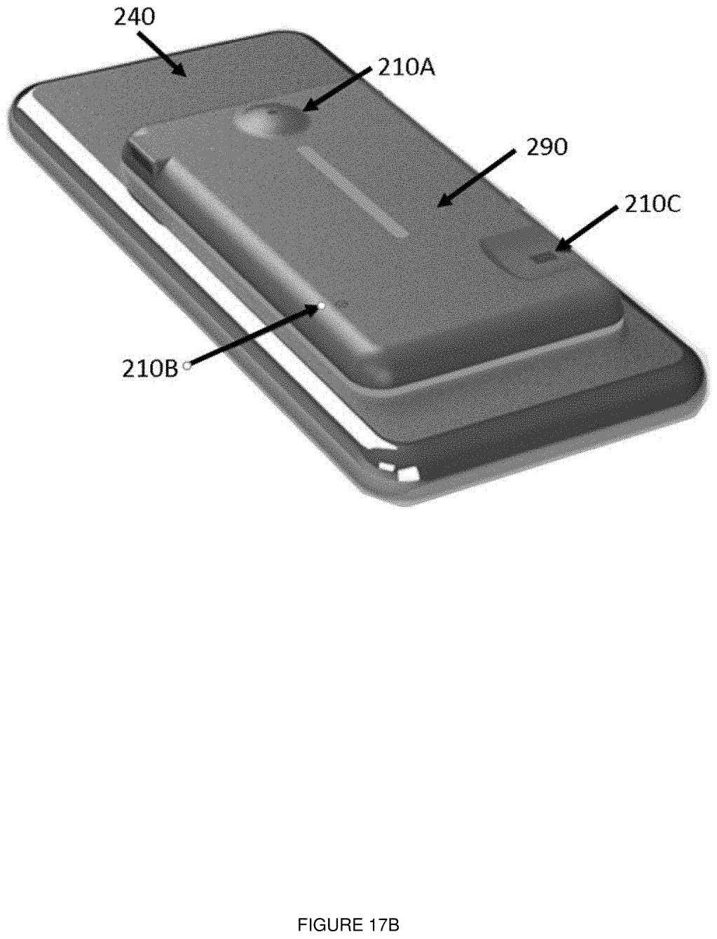

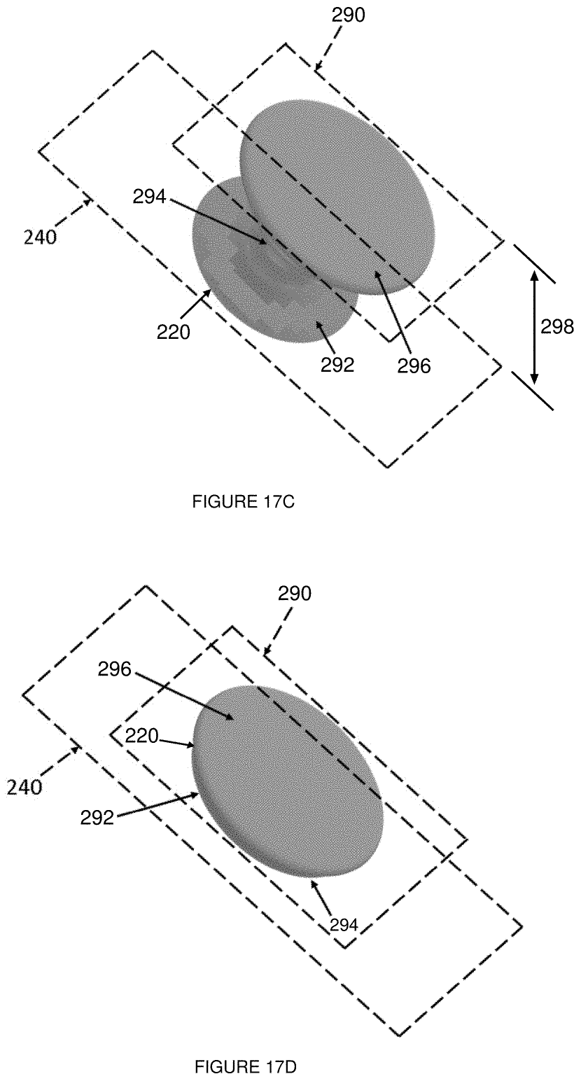

Adhesive section 220 may operate in a variety of modes. Examples of the different operating modes include double-sided tape, single-sided tape, temporary glues, hook and loop type fasteners, snap and lock type fasteners, or other types of adhesive mechanisms. In an alternative embodiment, the modular health platform may be integrated into the phone case to form a single unit (not shown). In some embodiments, the integrated modular health platform may snap and lock onto the mobile device. In other embodiments, the snap and lock mechanism may itself include adhesive section 220 or equivalent means on its device 240 side whereby the adhesive section 220 enables the integrated modular health platform to be securely attached to the device 240 by way of the mechanism. In one example, an accordion-like mechanism is coupled to the device 240 side of the integrated modular health platform where the accordion mechanism itself include adhesive section 220 on its device 240 side. Aspects of this example including the adhesive section 220 and the accordion-like mechanism may resemble features used in POPGRIPS by POPSOCKETS (www.popsockets.com, Boulder, Colo.), whereby a user of the device 240 may be provided with an ergonomic way to handle the device 240 with the integrated modular health platform during use (with the platform popped out from the device 240 as shown in FIG. 17C, further discussed below) thereof according to the present technology, and with the platform popped back in toward device 240 (as shown in FIG. 17D, further discussed below) when the platform is not being actively used. FIG. 2B includes modules 210, platform body 230, and modules adapters 240. Module adapters 240 may plug into modules 210 when modules 210 are inserted into platform 240. Modules adapters 240 can be USBC adapters or other types of adapters from transferring information.

In another embodiment, shown in FIG. 17A, each module 210 is a modular sensing entity coupled to a managed by a portable intermediate peripherals collection (IPC) device 245. In the example, the IPC device 245 manages each module 210 and plays a role of transmittance interface between the modules 210 and handheld device (e.g., mobile device 240). The IPC device 245 and modular sensing modules 210 (e.g., devices 210A, 210B, . . . , 210N) may be connected to each other via suitable wired and/or wireless communication components, or a mechanical, or a physical or an electronic port(s) capable of transmitting and receiving data communication signals using suitable protocols (e.g., ports 250A, 250B, . . . , 250N). As used herein, the term port means a device or component, a plurality of them, and/or a combination of the same, of the integrated modular health platform that functions in the present technology to provide a conduit for transmission and/or receipt of signals encoding data used in the disclosed systems and methods. Ports may include electronic and/or mechanical structures. As an example, a port for wired communication may include a receptable for a micro-USB cable's plug and associated electronics, circuitry and/or structures to provide for secure attachment so as to provide effective and reliable wired (e.g., serial) data transfer to and from the port as between two different devices, and/or within a single device, functioning in the present technology. As another example, a port for wireless communication may include a pair of radio frequency (RF) antennas and associated electronics, circuitry and/or structures to provide for RF signal transmission and/or receipt so as to provide effective and reliable wireless (e.g., BLUETOOTH) data transfer to and from the port as between two different devices, and/or within a single device, functioning in the present technology.

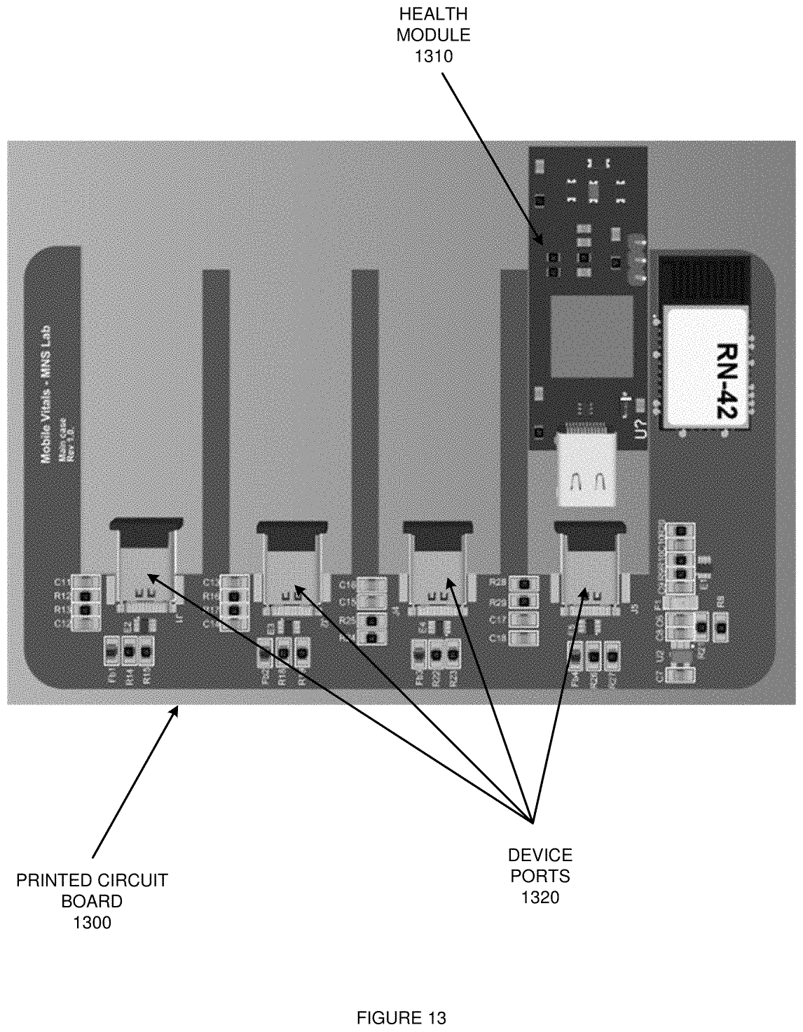

The IPC device 245 is configured to recognize the respective module(s) 210, alternately activate and deactivate the respective module(s) 210, provide electric power to the respective module(s) 210, initiate and mediate a suitable data transmission protocol, manage the incoming and outgoing data traffic between mobile device(s) 240 and the module(s) 210, and handle encryption of data between the detached/attached module 210. In the embodiment illustrated in FIG. 17A, module(s) 210 and IPC device 245 are attachable to mobile device 240 as a structurally integrated unit 255 as by, for instance, one or more mechanisms 260 enabling popping the unit 255 in and out to be convenient with each sensing posture. Unit 255 may include port(s) 250 in some embodiments. In an example, unit 255 may be embodied, at least in part, in a printed circuit board (PCB) with integrated port(s) 250, such as, or similar to, PCB 1300 described herein with reference to FIG. 13. Unit 255 may be enclosed by a housing 290 (also referred to herein as casing) for attachment to mobile device 240, as shown in FIG. 17B, for example, where three sensing modules 210 are capable of measuring vital signals and generating biosignals 260: temperature sensing module 210A, respiratory rate sensing module 210B, and heart rate & pulse ox sensing module 210C). In the embodiment of FIG. 17B, ports 250 (not visible within housing 290) are physical electrical ports 250 providing the functions describe above with reference to FIG. 17A. An application and/or memory devices present or otherwise in communication with components of mobile device 240 may thus receive data representative of vital signs (e.g., biosignals 260) of a patient from the IPC device 245 measured by each sensing module 210, and these data may be further processed and/or stored for various useful clinical and/or diagnostic ends including according to the embodiments of the systems and methods of the present disclosure.

In some embodiments, the integrated modular health platform (e.g., including housing 290, as shown in FIG. 17B) may be attached to mobile device 240 by way of an accordion-style mechanism, as shown in FIGS. 17C and 17D. In one example, the accordion-style attachment mechanism includes the adhesive section 220 on a device side of a base piece 292 for coupling piece 292 to a portion of mobile device 240. In another example, base piece 296 2ay be coupled to mobile device 240 by way of a mating structure on piece 292 and a portion of mobile device 240, which may enable the two structures to be securely, yet readily detachably, coupled to one another. The accordion-style attachment mechanism of FIGS. 17C and 17D may include a distal piece 296 for coupling to housing 290, for example. In one example, proximal piece 296 includes another adhesive section (similarly to as described herein for adhesive section 240) for coupling proximal piece 296 to housing 290. In another example, proximal piece 296 may be coupled to housing 290 by way of a mating structure on piece 296 and housing 290, which may enable the two structures to be securely, yet readily detachably, coupled to one another. The accordion-style attachment mechanism includes an accordion structure 294 shaped and configured to be alternately extended and retracted, as shown in FIG. 17 and FIG. 17B, respectively. In the extended position shown in FIG. 17A, the integrated modular health platform depicted by housing 290 in FIGS. 17A and 17B can be spaced from mobile device 240 by a distance 298. A maximum value of distance 298 is specified by dimensions of accordion structure 294. In some embodiments, distance 298 can be adjustable by a user in two or more values including the maximum distance 298 value. The extent to which distance 298 is adjustable is, again, dictated by the particular dimensions and configuration of accordion structure 294. Inclusion of the accordion-style mechanism in embodiments of the present technology may enable users to conveniently manipulate the modular integrated health platform and easily also utilize a smartphone screen, for instance, of mobile device 240, and vice versa. In this manner, users may be less prone to dropping their mobile device 240 having the attached modular integrated health platform which, among other benefits, may reduce the instances of malfunctions and enhance patient compliance with the prescribed use of the present technology.

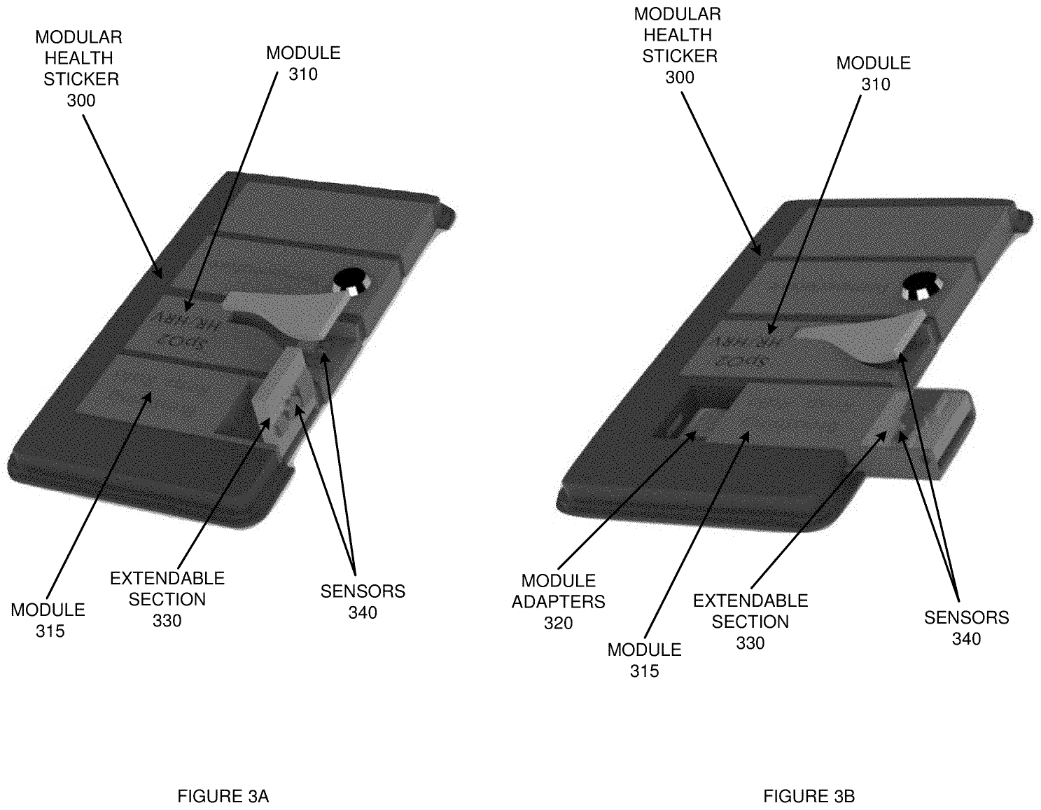

FIGS. 3A-3B illustrate examples of modules that may be applied to a modular health sticker according to one or more embodiments of the present technology. FIG. 3A includes modular health sticker 300, modules 310 and 315, extendable section 330, and sensors 340. FIG. 3B includes each element of FIG. 3A and additionally includes module adapters 320. Module 310 is configured to measure the heart rate and the SpO₂ of a patient while module 315 is configured to measure the breathing rate of a patient and are both embedded into health sticker 300. Health sticker 300 is not limited to only these types of modules. Module 310 and module 315 includes sensors 340 for the measuring a vital signs or other biometric signals of a patient. Module 315 includes extendable section 330 positioned adjacent to the exposed end of module 315. Extendable section 330 allows for the sensors 340 on module 315 to extend outwards away from modular health sticker 300.

In some embodiments, when modular health sticker is applied to a mobile device, one or more of the sensory elements of sensors 340 can be positioned on extendable section 330. Extendable section 330 may be pulled out and flipped forward as shown in FIG. 3B so that the one or more sensory elements of sensors 340 will face up in the same direction as the screen of the mobile device. When not in use, extendable section 340 may be folded down as shown in FIG. 3A to be stored in a compact position inside a module. Although some modules may have a functionality similar too, or the same as extendable section 340 that can be pulled out and flipped forward, other modules may lack this functionality. For instance, when a module can measure a vital sign without needing to be flipped forward, it can lack this portion.

Modules 310 and 315 can include module adapters 320 that communicatively connect modules 310 and 315 with health sticker 300. In some embodiments, the module adapters 320 transfer medical information relating to the measured vital signs of the user from modules 310 and 315 to modular health sticker 300. Examples of module adapters 340 include, but are not limited to, USBC adapters, micro-USB adapters, or other adapters for information transfer. In alternative embodiments, modules 310 and 315 may include Bluetooth functionality to transmit medical or other information wirelessly to modular health sticker 300 without the use of adapters 320.

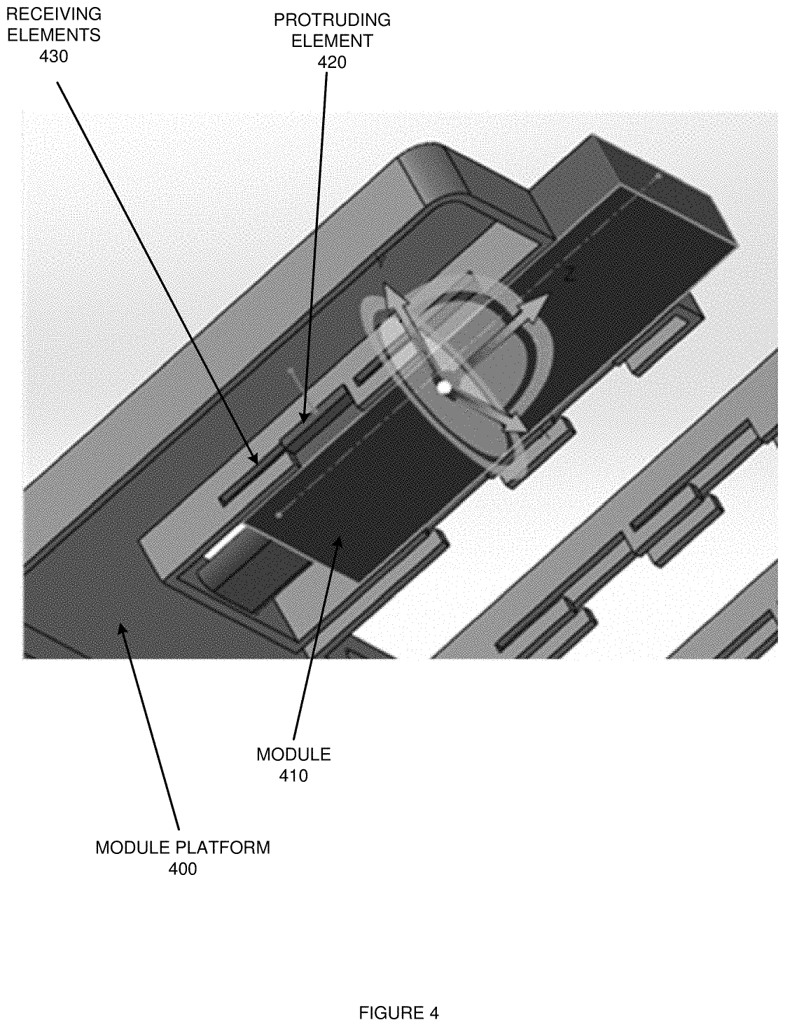

FIG. 4 illustrates an example of a module platform according to some embodiments of the present technology. As shown in FIG. 4, module 410 can be connected to the module platform 400 through a slide and lock feature. Module 410 can include connective protruding elements 420 that extend outwards from the main body of module 410. Protruding elements 420 can correspond to receiving elements 430 on module platform 400 that are configured to interlock with the protruding elements 420 of module 410. In some embodiments, module 410 may be pushed from the bottom of platform 400 to slide forward to interweave protruding elements 420 of module 410 with receiving elements 430 of module platform 400. In other embodiments, the module 410 and platform 400 can include external magnetic features to secure module 410 to platform 400 magnetically. Alternatively, module 410 and platform 400 may have a snap-and-lock feature where module 410 snaps into place when inserted into platform 400. It should be noted that the mode of attachment exhibited by module 410 and platform 400 are not limited.

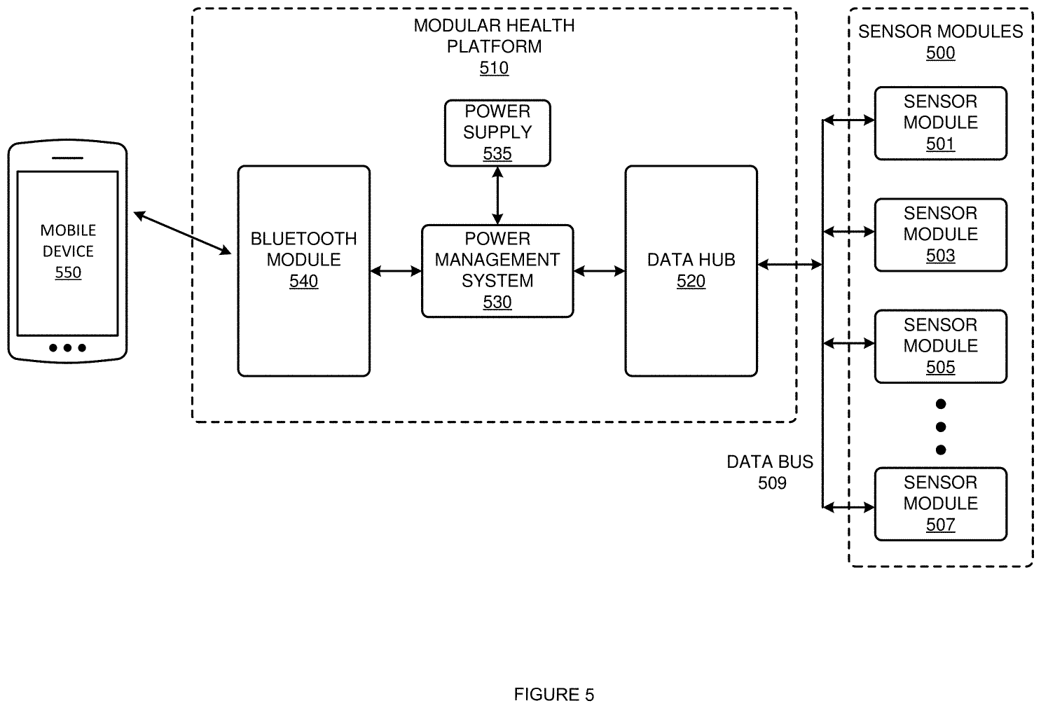

FIG. 5 illustrates a block diagram depicting an example of a modular health platform 520 and sensor modules 500 according to some embodiments of the present technology. In the embodiments illustrated in FIG. 5, the system can include sensor modules 500, modular health platform 510, and mobile device 550. Modular health platform 510 can include data hub 520, power management system 530, power supply 535, and Bluetooth module 540. Sensor modules 500 include sensor modules 501, 503, 505, and 507. However, it should be appreciated that the number or the type of sensor module of sensor modules 500 is not limited.

Data hub 520 is communicatively coupled to each of sensor modules 500 through data bus 509. Bluetooth module 540 provides a data connection between a mobile device 550 modular health platform 510. Data hub 520 can collect and aggregate sensor data from obtained from sensor modules 500. Power management system 530 can distribute power from power supply 535 (e.g. a rechargeable battery) to each component of modular health platform 510 as well as sensor modules 500. In some embodiments, modular health platform 510 can include a plurality of docking stations to hold sensor modules 500.

The docking stations can incorporate data bus 509 that connects sensor modules 500 to data hub 520. Each of sensor modules 500 are interchangeable and may connect to data bus 509 from any docking station. Data bus 509 is not limited by sensor type. Each sensor module of sensor modules 500 can measure the vital signs of the user and transmit these vital signs to data hub 520. Sensor modules 500 may connect to the data hub through a USBC type adapter or other type of data bus or data transfer adapter. Modular health platform 510 can include a plurality of adapters allowing for any number modules to connect to the modular health platform 510 simultaneously. Sensor modules 500 may communicate with data hub 520 through any type of commination protocol. Examples of communication protocols include serial peripheral interface (SPI) protocol, or inter-integrated circuit (I2C) protocol.

Data hub 520 communicates information received from sensor modules 500 to Bluetooth module 540. Bluetooth module 540 may form a wireless communicative link between data hub 520 and mobile device 550 or other types of computing devices. Examples of computing devices that may receive and/or transmit information to or from Bluetooth module 540 include mobile phones, smart phones, laptop computers, desktop computers, tablet computers, smart watches, or other computing devices with Bluetooth functionality. In alternative embodiments, data hub 520 may form a wired connection with a mobile device 550 or instead of linking to the mobile device 550 via Bluetooth.

Each sensor module of sensor modules 500 may include a unique sensor identification (ID) number. When any of sensor modules 500 connect to modular health platform 500, the module reports the sensor ID to data hub 520 signifying the module is available for use. Data hub 520 can include a sensor ID table that includes sensor IDs, the sensor functions associated with the sensor IDs, and the type of sensor associated with the sensor ID. Once the sensor ID is reported to data hub 520, data hub 520 can match the sensor ID with a corresponding sensor ID stored in the sensor ID table to identify the connected sensor module.

In some embodiments, the sensor ID table may be stored on mobile device 550 and mobile device 550 may identify the senor ID. When a mobile device 550 connects to modular health platform 510, data hub 520 reports to mobile device 550 the types of sensor modules and services that are available. In various embodiments, power management system 530 can control the power supply to each component of the modular health platform 510 and can selectively turn on or off the power supply to each sensor module based on user input. As a new module is inserted into or removed, modular health platform 510 informs the mobile device 510 about the availability or unavailability of the sensor module.

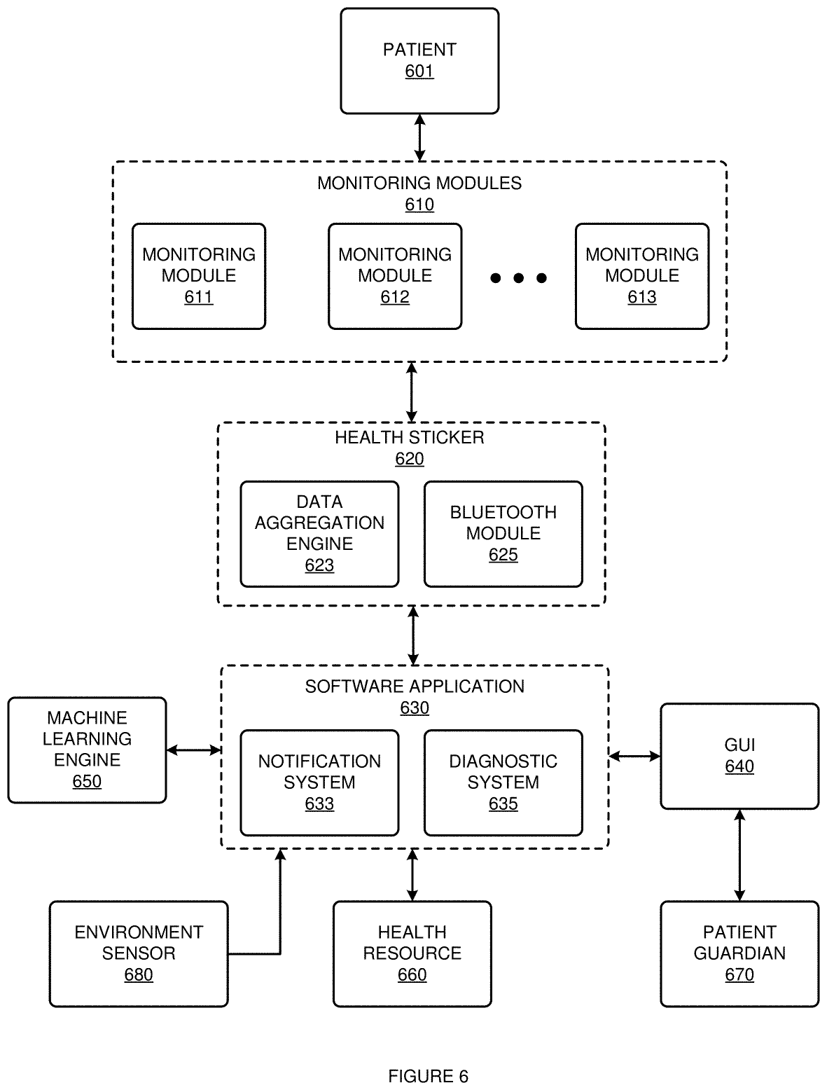

FIG. 6 illustrates an example of a system architecture for monitoring the health condition of a user according to one or more embodiments of the present technology. The system can include monitoring modules 610. Monitoring modules 610 may further include monitoring modules 611, 612, and 613 and is communicatively coupled to health sticker 620. In some embodiments monitoring modules 610 can monitor one or more bodily functions of patient 601. Each monitoring module of monitoring modules 610 can include a plurality of sensors that measure the one or more bodily functions of patient 601. For example, monitoring module 611 may include multiple sensors and may measure more than one bodily function of patient 601. Monitoring modules 610 can report the measured bodily functions to health sticker 620.

Health sticker 620 can includes data aggregation engine 623 and Bluetooth module 623. Data aggregation engine 623 may ingest and then interpret the measurements collected by monitoring modules 610. In some embodiments, data aggregation engine 623 interprets the measurements by fusing the measurements collected by one or more of monitoring modules 611, 612, and 613 to calculate metrics relating to the bodily functions. For example, monitoring module 611 may report multiple measurements (e.g., MIC, air pressure, air temperature) and data aggregation engine may then fuse the multiple measurements to calculate medical information relating to the multiple measurements (e.g., a breathing rate). In some embodiments, data aggregation engine 623 algorithmically fuses these measurements to calculate the medical information.

After interpreting the measurements collected by monitoring modules 610, data aggregation engine 623 relays the medical information to Bluetooth module 625 which intern, transmits the medical information to software application 630. Software application 630 includes notification system 633 as well as diagnostic system 635. Software application 630 may operate on a computing device, mobile computing device, or on a similar platform. Software application 630 is operatively coupled to graphic user interface (GUI) 640 and communicatively coupled to machine learning engine 650, health resource 660, and environment sensor 680. In some embodiments, software application 630 visually displays the medical information received from Bluetooth module 630 on GUI 640 in real time. The visual display may include one or more plots, charts, informatic graphics, or tables. The visual display can update in real time to reflect changes in the medical information. For example, if the heart rate of patient 601 is measured, a change in the heart rate of patient 601 would result in a change the visual display on GUI 640. Software application 630 may additionally transmit the medical information to machine learning engine 650 and health resource 660.

In further embodiments, notification system 633 may track changes in the medical information as software 630 receives medical information. Notification system 633 can generate an alert or notification in response to changes, or the lack thereof, in the medical information. For example, a sudden drop in heart rate may trigger the generation of an alert. Alerts generated by notification system 633 may be presented on GUI 640 where patient guardian 670 or patient 601 can view the alert. The type of alert generated by notification system is not limited and may be any type of audio or visual alert. Notification system 633 may be further configured to notify patient 601 to take any number of actions. For instance, notification system 633 may generate a notification informing patient 601 to take a prescribed amount medication. Notification system 633 may additionally track when and if patient 601 or patient guardian 670 responded to the action notification.

Diagnostic system 635 of software application 630 assists in the identification of a state of a medical condition of patient 601. Diagnostic system 633 may include one or more interactive questionnaires (not shown) that include questions relating to the medical condition of patient 601. The questions may ascertain one or more symptoms of patient and may identify the medication adherence of patient 601. The interactive questionnaire may be presented to patient guardian 670 or patient 601 via GUI 640. Upon completion of the questionnaire by patient guardian 670 or patient 601, diagnostic system 635 scores the answers provided by patient 601 to develop a health score for the user. Diagnostic system 635 can augment the health score based on the measured vital signs of patient 601. The health score can dictate, at least in part, the severity level of the current state of the medical condition of the user.

Diagnostic system 635 may further include one or more action plans (not shown) for treating the medical condition of the user. The action plans are provided by health resource 660 or a medical professional associated with patient 601 and may be presented to the patient guardian 670 or patient 601 via GUI 640. The action plans can include one or more levels, where each level indicates a severity of the medical condition of patient 601 and a recommended course of action to address the medical condition of patient 601. For example, one level may indicate a minor severity in the medical condition of patient 601 while a different level may indicate a moderate severity in the medical condition of the user. Each level in the action plan includes a prescribed response to the current severity of the medical condition. Diagnostic system 635 can select a level in an action plan based on the health score provided by the health questionnaire. Diagnostic system 633 may also use environmental data obtained from environment sensor 680 when selecting a level in the action plan. Furthermore, diagnostic system 635 may select a level in the action plan based on information received by machine learning engine 650 and health resource 660.

Machine learning engine 650 may be configured to ingest the medical information received by software application 630. In some embodiments, machine learning engine 650 algorithmically processes the medical information to determine a current state in the medical condition of patient 601. In determining the current state of the medical condition, machine learning engine 650 may assign a machine learning health score to the current state of the medical condition. Once a machine learning health score is generated, machine learning engine 650 can send the score to diagnostic system 635. The machine learning health score can correspond with a severity level of the action plans of diagnostic system 635. In other embodiments, machine learning engine 650 may be configured to ingest the health score, questionnaire answers, and environmental information obtained by diagnostic system 635. Machine learning engine 630 can then algorithmically combine the health score and medical information of diagnostic system 635 with the machine learning health score to generate a combined heath score and then transmit the combined health score to diagnostic system 635. Diagnostic system 635 may then use the combined health score when selecting a level in the action plan.

Health resource 660 is representative of an access point for one or more medical professionals, associated with patient 601, to monitor the health of patient 601. Health resource 660 can receive medical information from software application 630 as well as updates on the current state of the medical condition of patient 601. In some embodiments, health resource 660 can instruct notification system 633 to alert patient 601 to take any number of prescribed actions.

Environment sensor 680 is representative of one or more sensors that collect environmental information that may affect the health of patient 601. Environmental information can include pollution levels, pollen levels, UV levels, epidemic severity, or other information pertaining to the health of patient 601.

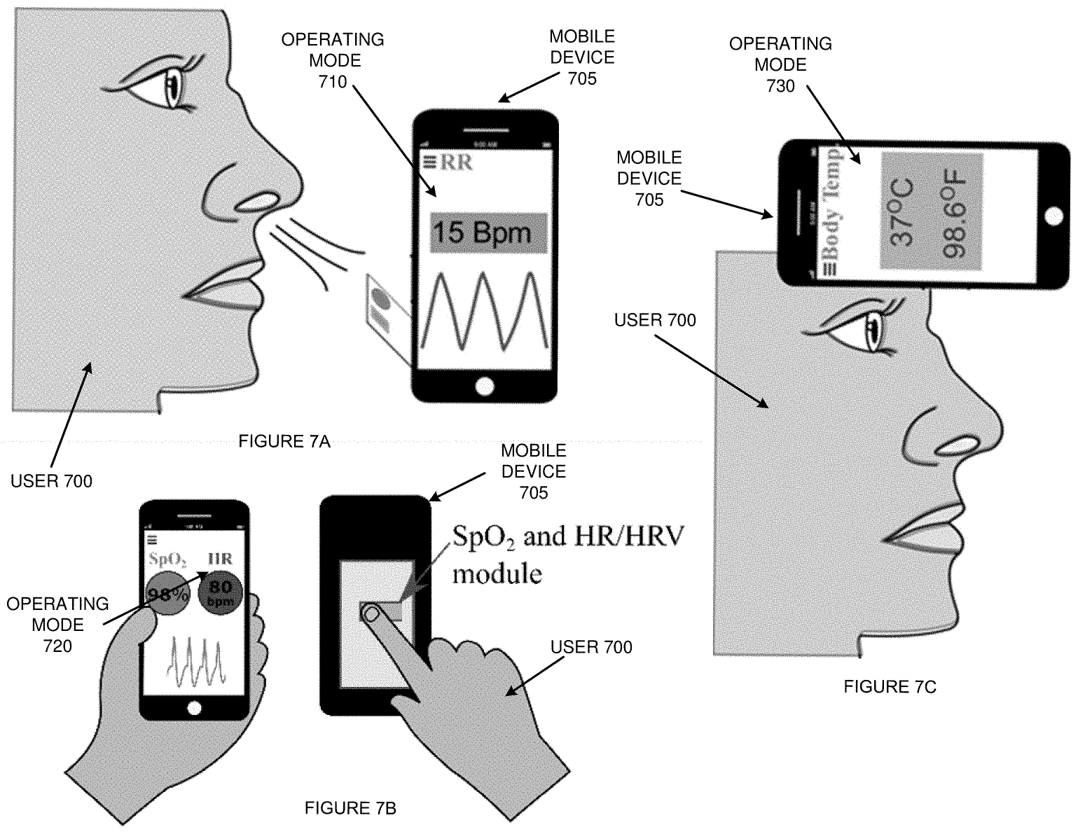

FIGS. 7A-7C are diagrams showing examples various operating modalities of a health monitoring platform according to one or more embodiments of the present technology. FIGS. 7A-7C include user 700 and mobile device 705. FIG. 7A includes operating mode 710 for measuring the breaths per minute (BPM) of a user 700 and displaying the measured information on a mobile device 705. Operating mode 710 may track the respiratory rate, the respiratory variability, the respiratory pacing, and respiratory pattern of user 700.

FIG. 7B includes operating mode 720 for measuring oxygen saturation levels (SpO₂) and the heart rate and heart rate variability of user 700 and displaying the measured information on mobile device 705. FIG. 7C includes operating mode 730 for measuring the body temperature of user 700 and displaying the current body temperature of user 700 on the mobile device 705. The health monitoring platform can integrate into a smartphone and detect a wide variety of health metrics. In each of FIGS. 7A-7C, the measured biometric information is presented on the screen of mobile device 705 in real time as user 700 interacts with the chosen mode of operation.

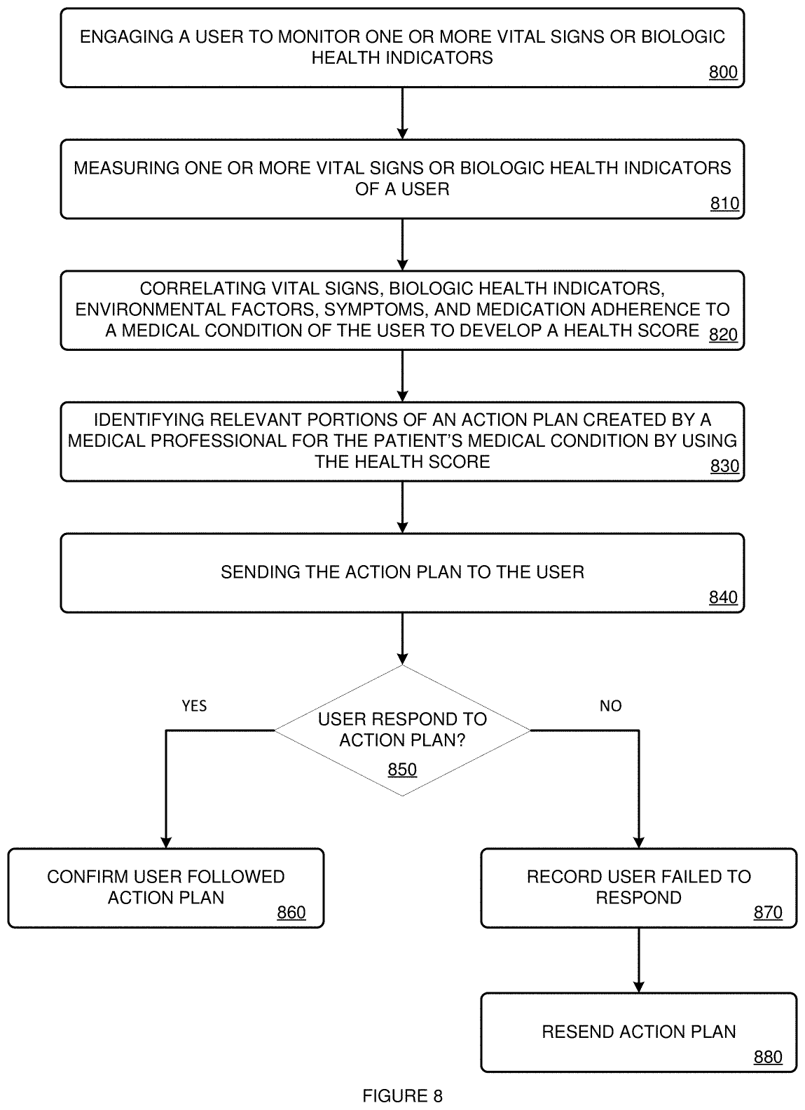

FIG. 8 is a flowchart depicting an example of a method of operating a modular health platform according to some embodiments of the present technology. Process step 800 includes engaging a user to monitor one or more vital signs of biologic health indicators. Engaging the user may involve sending the user one or more text message notification indicating the user needs to monitor vital signs or biologic signals. The text message notifications can include incentives to encourage the user to being monitoring the required vital signs. Process step 810 includes measuring one or more vital signs of a user. Measuring the vital signs of a user can include using one or more sensor modules equipment on a modular health platform. The measured vital signs of the user may relate to a health condition of the user. Process step 820 includes correlating vital signs, biologic health indicators, environmental factors, symptoms, and medication adherence to a medical of the user to a medical condition of the user to develop a health score. In some embodiments, the health score indicates the current state of a medical condition of the user. Correlating the vital signs of the user to the medical condition may include processing the vital sign measurements with a machine learning engine to generate a health score. Additionally, correlating can include presenting the user with a questionnaire that includes questions relating to the user's current symptoms and medication adherence and then processing the user's answers to develop the health score.

Process step 830 includes identifying relevant portions of an action plan created by a medical professional for the patient's medical condition by using the health score and medically indicated actions found in the literature. In some examples, identifying the relevant sections of the health score may include populating the action plan with medical information relating to the patient's current medical condition. The data may indicate portions of the action plan that are of primary importance and portions of the action plan that are of secondary importance. The action plan may have multiple sections where each of the multiple sections corresponds to a different health score.

Process step 840 includes sending the action plan to the user. Sending the action plan may include presenting the user with an in-app notification. Decision block 850 includes determining the user responded to the action plan. This can include monitoring if the user responded to the notification or can include receiving positive affirmation from the user that the action plan was received. If the user responded to the action plan, the method continues with process step 860 which includes confirming that the user followed the action plan. In some examples, confirming can be done by sending a confirmation request to the user. However, if the user failed to respond to the action plan, the method continues with process step 870 that includes recording user failed to respond followed by process step 880 which includes resending the action plan and or notification of other (parent or health care resource).

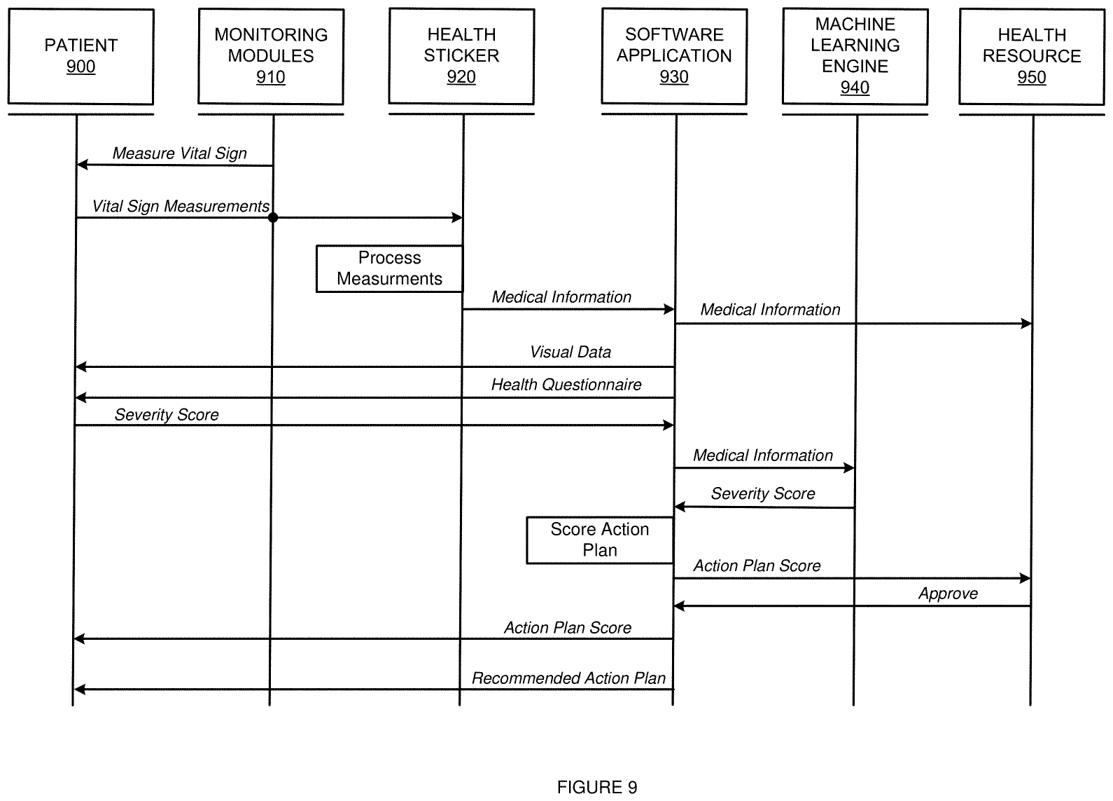

FIG. 9 is a sequence diagram depicting an exemplary method of operation for a modular health monitoring system according to one or more embodiments of the present technology. Monitoring modules 910 monitor one or more vital signs of patient 601. This may include recording various health metrics such as breathing rate or heart rate. Monitoring module 910 then receives vital sign measurements from patient 900 and transmits the vital sign measurements to data health sticker 920. Health sticker 920 then processes the vital sign measurements to create medical information describing the vital sign measurements. In some embodiments, health sticker 920 algorithmically combines multiple vital sign measurements to create a single measurement and then reports this single measurement as medical information. Software application 930 may also assist health sticker 920, in some embodiments, in combining vital signs and/or other measurements. For example, a SpO₂/HR module activated in health sticker 920 can calculate all the values on the chip and return the value to software application 920 for displaying. However, the RR needs to convert the raw signals from the hardware to the measurement on the software side.

Once data health sticker 920 finishes processing the vital sign measurements, health sticker 920 the sends the medical information to 930. Upon receiving the medical information, software application 930 stores the medical information, copies the medical information, and then distributes the copied medical information to medical resource 950. Software application 930 may then present the medical information as visual data to patient 900. The visual data may include plots or graphs depicting the medical information. In some examples, the visual data is updated in real time in response to changes in the measured vital signs. Software application 930 may additionally send a health questionnaire to patient 900 that includes questions relating to the current state of a medical condition of patient 900. Patient 900 can then answer the health questionnaire and the answers provided by patient 900 form a severity score describing the severity of the current state of the medical condition. In some embodiments, local environmental data (e.g., pollen count or scores) can be received and processed by software application 930.

Software application 930 then transfers the medical information, or at least a copy of the medical information, to machine learning engine 940. Machine learning engine 940 analyzes the medical information to predict the severity of the medical condition. Machine learning engine 940 can then develop a severity score representative of the current state of the medical condition and send this severity score to software application 930. Software application 930 can then combine the severity scores received from machine learning engine 940 and patient 900. In some embodiments, the combined scores are used by software application 930 to highlight relevant portions of an action plan provided by health resource 950. The action plan score can be associated with a prescribed respond to the current state of the medical condition of patient 900. Once the action plan score is generated, software 930 transfers the action plan score to health resource 960 for approval. Health resource 660 may then notify software application 930 that the action plan score is approved. Once approved, software application 930 may present the action plan score to patient 900 and send a recommended action plan provided by health resource 950 to address the current state of the medical condition to patient 900.

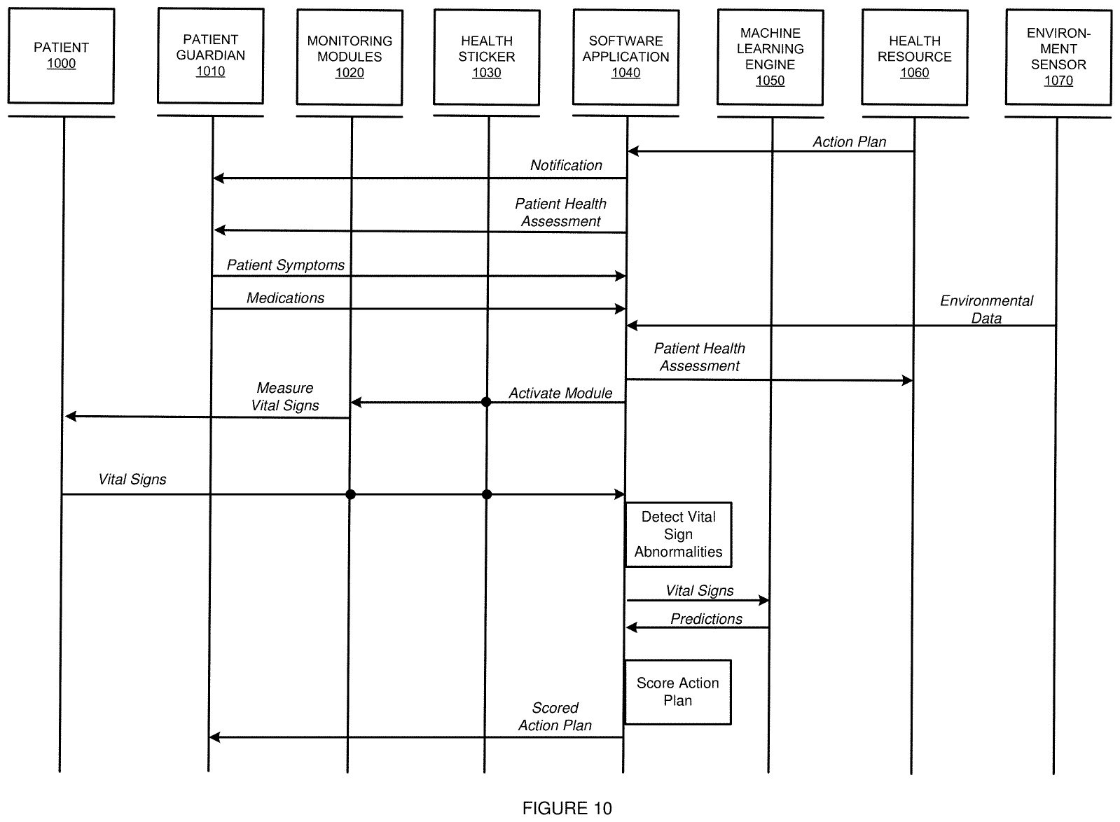

FIG. 10 is a sequence diagram depicting an exemplary method of operation for a modular health monitoring system according to one or more embodiments of the present technology. In some embodiments, health resource 1060 transfers an action plan to software application 1040. The action plan may be developed by health resource 1060 or by a medical professional associated with health resource 1060 for treating a medical condition of patient 1000. Once in possession of the action plan, software application 1040 sends a notification to patient guardian 1010 and may determine if patient guardian 1010 responds to the notification. After notifying patient guardian 1010, software application 1040 sends a patient health assessment to patient guardian 1010. The patient health assessment may include questions relating to the symptoms of patient 1000 and the medication adherence of patient 1000. Patient guardian 1010 may complete the patient health assessment on behalf of patient 1000 and transmits the responses to software application 1040. Software application 1040 collects environmental data from environmental sensor 1080. Once the environmental data is collected, software application 1040 can transfer the patient health assessment, or a copy of the patient health assessment, to health resource 1060 for further evaluation.

In some embodiments, software application 1040 may then direct health sticker 1030 to activate monitoring modules 1020. Monitoring modules 1020 can measure one or more vital signs or other biological indicators of patient 1000. The vital signs measured by monitoring modules are then relayed back to health sticker 1030 which transfers the vital sign measurements to software application 1040. Software application 1040 may then review the measured vital signs by checking the vital signs against established medical literature to determine if any of the vital signs of patient 1000 are abnormal. If the vital signs of patient 1000 are determined to be abnormal, software application 1040 may transfer the vital sing measurements to machine learning engine 1050 to confirm the abnormality. In some embodiments, software application 1040 may also notify health resource 1060 of the abnormality.

Machine learning engine 1050, upon receiving the vital sign measurements and other medical information, will ingest and process the vital sign measurements and other medical information to form predictions on the current state of the medical condition of user 1040. The predictions are then sent back to software 1040. Utilizing the predictions from machine learning engine 1050 as well as information obtained from the health assessment and the environmental data, software application 1040 can score the action plan. Scoring the action plan can include indexing the action plan to identify relevant portions of the action plan for responding to the current state of the medical condition of patient 1000. Software application 1040 can then transfer the scored action plan to patient guardian 1010.

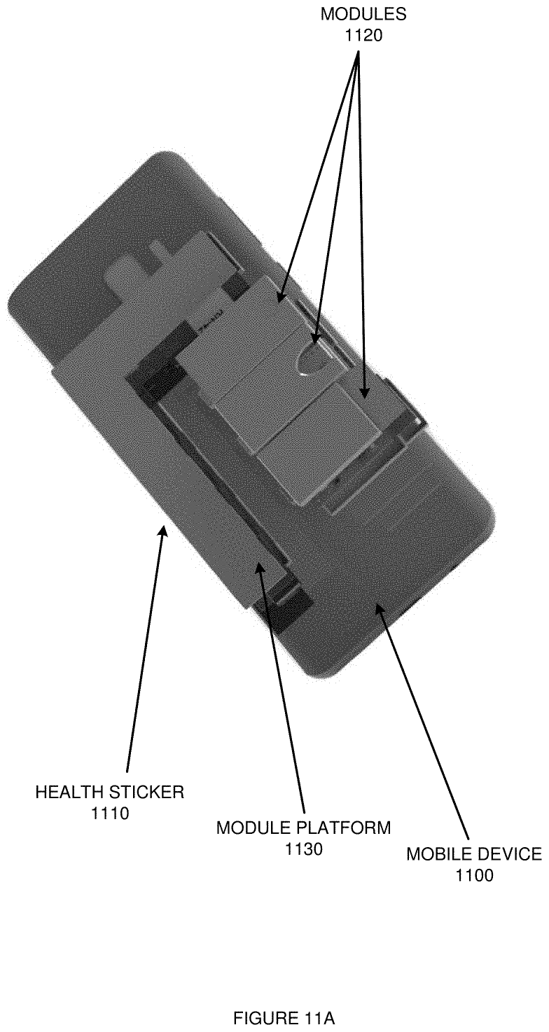

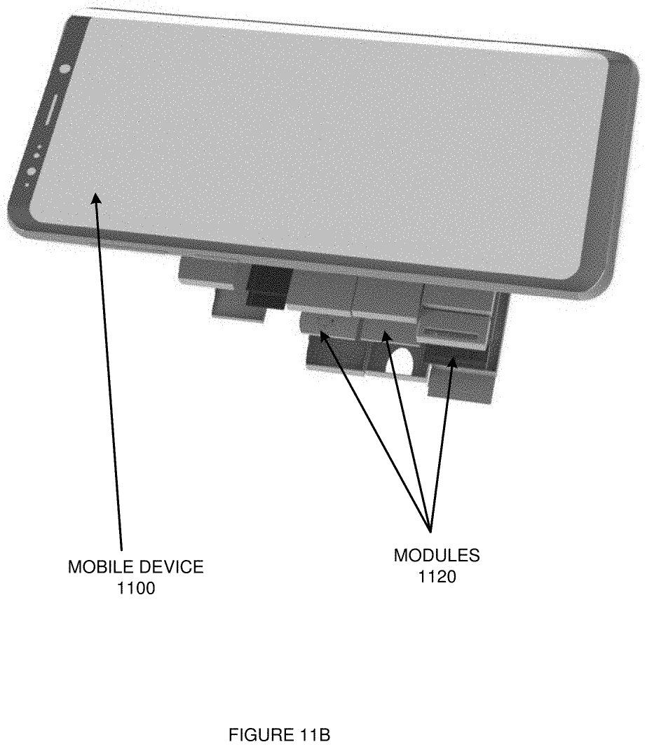

FIG. 11A illustrates a modular health sticker according to one or more embodiments of the present technology. FIG. 11A includes health sticker 1110 which further includes modules 1120 and module platform 1130. In some embodiments, module platform 1130 adheres to the reverse side of mobile device 1100. Modules 1120 may measure biometric information of a user and upload this biometric information to module platform 1130. In some embodiments, module platform transmits the measured biometric information to mobile device 1100. Modules 1120 are positioned in such a way that allows a user to operate one or more of modules 1120 while simultaneously operating mobile device 1030. Biometric information collected by modules 1010 that is transmitted to mobile device 1030 may be displayed on the screen of mobile device 1030.

FIG. 11B illustrates the modular health sticker 1100 in relation to mobile device 1100 from a different angle than that of FIG. 11A according to some embodiments of the present technology. Modules 1120 are positioned near the periphery of mobile device 1100 to allow for a user to access modules 1120 while viewing the screen of mobile device 1100. In some embodiments, modules 1010 include a sliding feature allowing the module to extend outwards from module platform 1020. The extended module of modules 1010 can then be readily operated while the screen of mobile device 1030 faces the user.

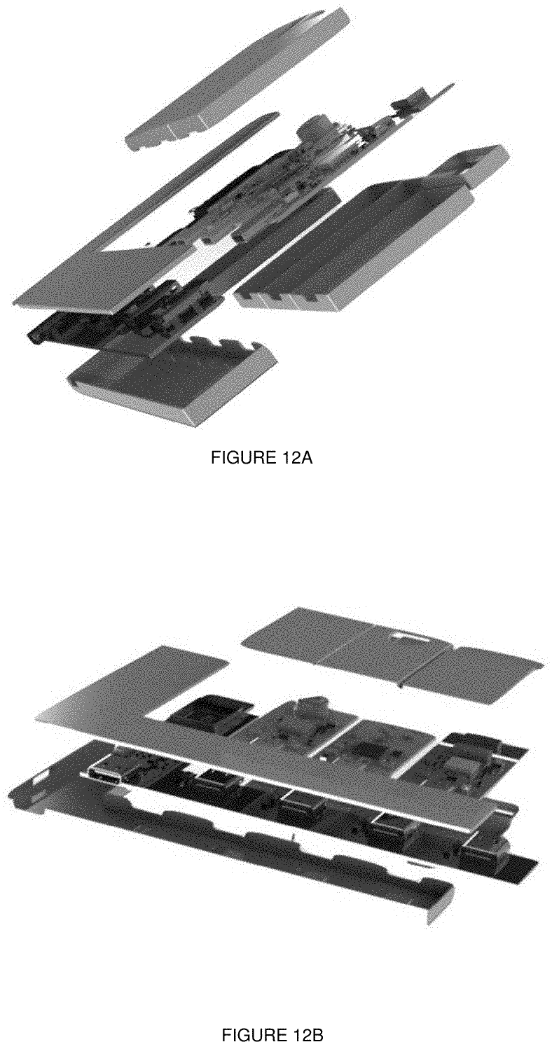

FIGS. 12A-12B present an expanded 3D model a modular health sticker 1200 according to more or more embodiments of the present technology. Modular health sticker 1200 includes further includes module casing 1210 and internal components 1215. Internal components 1215 may include one or more elements a Bluetooth module, a data aggregation engine, and medical monitoring modules. In accordance with various embodiments, the main circuits can be protected by a soft cover, which can be made from silicone/rubber to absorb the force during the impact efficiently. Also, the material can be wipable, which is suitable to be used in the clinic.

A locking mechanism can be used to help firmly secure the circuit and allow for easy replacement of the soft cover. This can allow for different colors or designs. The top piece can have multiples holes to connect with the poles on the bottom part. The poles can also go through the holes that are punched on the circuit. This design helps to stabilize the circuit inside the case while in use. In some embodiments, the dimension of the main piece can be 110 mm×41 mm×5 mm and each module dimension may be 40 mm×20 mm. Other embodiments may have different sizes and dimensions. The height of each module can vary depending on the size of the sensors and other components.

Some embodiments of the breathing module can have a specific design that separates the sensor piece with the base. The two pieces can then be connected together using a flexible cable. The sensor can be protected inside a case which can be rotated inside the base. The design helps users to watch their breathing behavior by looking at the screen while monitoring. To do so, they rotate the sensor piece to expose the sensors out, then holding the phone and breath to the sensors while their eyes still look at the phone screen. After finishing the measurement, they can roll back the sensor piece to attach it back to the base. The base will contain all the necessary electronic components (see, e.g., FIG. 3).

The SpO₂ module case can be designed to expose the area of the light sensor where users place their fingers on top. To prevent the ambient light for distorting the signals, a rubber cover on top can be added to shield the finger and light sensor (e.g., as shown in FIG. 3).

FIG. 13 illustrates an example of a PCB according to one or more embodiments of the present technology. FIG. 13 includes PCB 1300 which includes health module 1310 and device ports 1320. The modules 1310 and associated ports 1320 are positioned on a single PCB 1300. In one embodiment, PCB 1300 may include components (e.g., input ports, regulators, power converters, protection circuitry, and the like, not shown in FIG. 13) for receiving electric power from a power supply (also not shown in FIG. 13) and delivering the electric power to components like modules 1310 in needed thereof. In one or more embodiments, health module 1310 can be used to measure biometric signals of a user. When collecting biometric information, health module 1310 transfers the biometric information via device ports 1320. Health module 1310 is not limited by the device port which it is inserted into and may operate with any of device ports 1320. In some embodiments, printed circuit board 1300 may be similar to, or the same as internal components 1215.

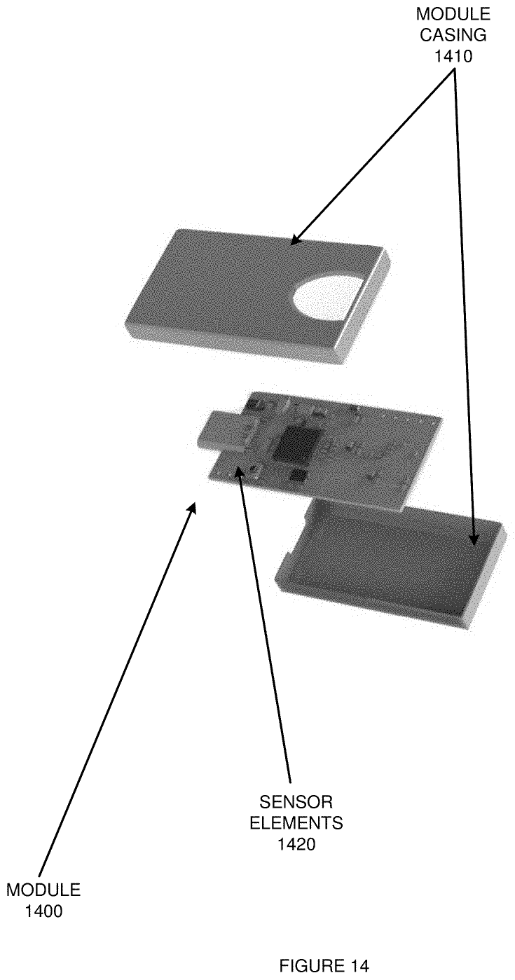

FIG. 14 illustrates a modular medical monitoring device according to some embodiments of the present technology. FIG. 14 include module 1400, module casing 1410, and sensor elements 1420. In some embodiments, module casing 1410 partially encases sensor elements 1420 to allow for sensor elements 1420 to measure one or more biometric signals while protecting components of sensory elements 1420 that do not need to be exposed. Casing 1410 may be metallic, molded plastic, or 3D printed plastic, or any other material that sufficiently protects sensory elements 1420 without interfering in the function of sensor elements 1420. Sensor element 1420 may include one or more sensors for measuring biometric signals of a user. Sensor element 1420 may include one or more sensors for measuring biometrics related to a health condition of a user. For example, if a user is a diabetic, sensor element 1420 may include sensors for measuring blood glucose levels. It should be appreciated the sensor elements 1420 are not limited by sensor type.

### Exemplary Machine Learning Overview

Aspects and implementations of the systems and methods of the present disclosure have been described in the general context of various steps and operations. A variety of these steps and operations may be performed through machine learning methods or may be embodied in a machine learning engine (e.g. machine learning engine 109). In accordance with some embodiments and for the purposes of explanation, an example implementation of a machine learning engine for measuring the severity of asthma in patients is provided.

Manual clinical scoring systems are the current standard used for acute asthma clinical care pathways. No automated system exists that assesses disease severity, time course, and treatment impact in pediatric acute severe asthma exacerbations. A machine learning applied to continuous vital sign data provides a novel pediatric-automated asthma respiratory score (pARS) by using the manual pediatric asthma score (PAS) as the clinical care standard.

Intermittent continuous vital sign monitoring data (heart rate, respiratory rate, and pulse oximetry) can be merged with the health record data including a provider-determined PAS in children between 2 and 18 years of age admitted to the pediatric intensive care unit (PICU) for status asthmaticus. A cascaded artificial neural network (ANN) may be applied to create an automated respiratory score and validated by two approaches. The ANN was compared with the Normal and Poisson regression models.

Out of an initial group of 186 patients, 128 patients met inclusion criteria. Merging physiologic data with clinical data yielded over 37,000 data points for model training. The pARS score had good predictive accuracy, with 80% of the pARS values within two points of the provider-determined PAS, especially over the mid-range of PASs (6-9). The Poisson and Normal distribution regressions yielded a smaller overall median absolute error.

The pARS reproduced the manually recorded PAS. Once validated and studied prospectively as a tool for research and for physician decision support, this methodology can be implemented in the PICU to objectively guide treatment decisions.

The use of pediatric clinical scoring systems in clinical care and research has exploded in the last decade. Clinical scoring systems appeal to multiple stakeholders because they are quantitative, can be validated and improve patient outcomes. Pediatric asthma is no exception; as the most common chronic disease of childhood, development of clinical scores and guidelines have helped to streamline and improve pediatric asthma care delivery.

Many hospitals have developed clinical care guidelines for management of acute asthma exacerbations built around manual, provider-determined asthma severity scores. Examples include the pediatric asthma score (PAS) and pediatric respiratory assessment measure. These scores contain similar elements but are customized to individual hospitals. The use of these scores to guide inpatient treatment of acute asthma exacerbations has improved patient outcomes including reduced length of stay, decreased admission rates, and decreased medication burden in both the emergency department and inpatient wards.

Respiratory scores like PAS contain subjective elements like auscultation, have limited interrater reliability, and are less sensitive in older children and adolescents. They are used intermittently and depend on frequent reassessment, increasing the burden on staff. The provider-determined PAS score used includes qualitative measurements. Despite being useful on the wards, PAS scores are often measured inconsistently and not regularly used to make care decisions.

There is significant variation in care of pediatric intensive care unit (PICU) patients with asthma, both within and across centers and hospitals. Despite the existence of stepwise guidelines for out-patient asthma management, there are no national or international guidelines to direct severe acute asthma exacerbation management in the PICU. Validated scores for asthma severity can be useful in clinical and quality research on asthma therapies such as intravenous bronchodilators and use of noninvasive ventilation.

Machine learning engines can generate an automated acute asthma severity score. Machine learning may be built on a foundation of mathematics, logic, probability, neuroscience, and decision theory. These foundational building blocks can be used to generate computer algorithms that can keep a record of the relative strength of associations between data elements (similar to memory) through repeated training sessions. As a result, machine learning can identify patterns in complex data and then uses those patterns to construct models that can predict outcomes without relying on explicit human-generated programming code. Supervised learning is a subfield of machine learning based on tools of classification and regression and may depend on the input of a labeled training data set to help the computer “learn” relationships. That knowledge is then applied to an independent testing data set. The accuracy of outputs can then be analyzed. Artificial neural networks (ANNs) are a specific set of algorithmic processes modeled after the structure of the human brain, designed to cluster and classify data and subsequently produce novel outputs.

The application of machine learning engines to passively collected vital sign data (e.g. heart rate, respiratory rate, and oxygen saturation) in critically ill pediatric asthma patients can generate a pediatric-automated asthma respiratory score (pARS) that could eventually replace PAS. Once created, the pARS can be validated and applied prospectively in the PICU, wards and emergency department to aid in clinical research and provider decision support without increasing the burden of staff or utilizing subjective measures.

The machine learning engine can be applied to a variety of patients to develop a pARS for each patient. Inclusion criteria included patients admitted to the PICU age 2 to 18 years old with diagnosis codes for status asthmaticus across all severities. Data from patients who had international classification of diseases (ICD) 9/ICD 10 procedure codes within the encounter for intubation, or received continuous invasive mechanical ventilation were excluded. Diagnosis codes for other potentially confounding chronic respiratory and neurologic conditions also disqualified patients (Table S1 and S2).

Demographic variables and time-stamped clinical data including charted PAS score, respiratory support, and medications were obtained from the electronic health record (EHR). Using bed numbers and admission/discharge time stamps, each patient's continuous vital sign information was manually extracted from a central research database. This database stores vital sign data (numeric and wave-form) from PICU patients attached to the Phillips monitors. Data extracted for this study included time-stamped values for heart rate, respiratory rate, and pulse oximetry.

The EHR respiratory flow sheet data were aligned with vital sign data using date and time stamps for PAS score in MATLAB. The recorded PAS was used as an outcome to train the supervised machine learning models. Patient data with complete records of three parameters (heart rate, respiratory rate, and pulse oximetry readings) overlapping with PAS time points were included. Patient data with incomplete alignment were excluded to create the final study cohort of 128 patients. For each PAS score recorded in the medical record, 20 minutes of vital sign measurements were associated with one PAS score via a standard one-to-many matching strategy to create discrete time periods for machine learning engine training. To control for age-based variability in heart rate and respiratory rate, z-scores for each patient's heart rate and respiratory rate were calculated using the patient's age and normalized percentile. To exclude readings likely due to artifact, thresholds for heart rate, respiratory rate, and oximetry values were also applied.

Patients were randomly assigned into a training (80%) or testing (20%) set (balanced validation) and compared with ensure balance by demographic criteria and clinical criteria. A separate randomized 10-fold cross validation was conducted to further validate findings.

Supervised machine learning techniques are use and output data to find patterns and make predictions. A cascaded ANN was used to predict a respiratory score ranging from 1 to 15, based on training inputs. Neural networks depend on linking “neurons” or multiple learning units to detect patterns in data. In comparison what conventual feed-forward neural network, the cascaded network structure is more advanced. It augments a set of cascaded paths to direct the nodes in the preceding and current layers to be input into the next layer. The cascaded ANN included with hidden layers with 3 to 50 neurons in each layer. Machine learning regression models based on normal and Poisson distribution were used for comparative purposes. The accuracy of machine learning models was assessed by comparison of the median absolute error (MAE) for each of the testing sets.

In the training data, there were 4943 original PAS scores, or 12.5 manual scores calculated per day of hospitalization. The median (range) of PAS scores was 7 (5-15). Plots of PAS score vs heart rate z-score, PAS score vs respiratory rate z-score show a positive relationship.

The training (n=102) and testing (n=26) patients in the balanced set showed comparable distributions of demographic and clinical factors. The balanced training set (n=102 patients and 37,084 data points) was reduced slightly to 36,321 data points after application of the artifact thresholds for age-adjusted heart rate and respiratory rate. For the 10-fold cross-validation, the number of data points included in each fold of training varied from approximately 34,000 to 38,000.

On the basis of the comparison of MAE for the balanced testing set for each of the machine learning models, the cascaded ANN with eight hidden layers trained with the balanced group yielded the smallest MAE of 1.21. The MAEs across the balanced group Poisson and Normal models were 1.24 and 1.25, respectively. The Poisson and Normal models each yielded slightly higher MAEs for the extreme values of the PAS scores. The most accurate predictions occurred in the mid-range values of 6-9, where the most training data existed.

In asthma clinical care guidelines, 2-point discrimination on the PAS scale is a clinically relevant range for guiding care. Thus, the predictability of pARS was evaluated in the ±2 point range. Specifically, 80% of the pARS scores produced by the ANN of the machine learning engine were within ±2.10 of the recorded PAS for the balanced testing set. The results from the 10-fold cross validation are similar. The pARS values are also aligned well with PAS when mapped over time across the course of individual patient encounters.

The machine learning engine produced pARS, a novel, pediatric-automated asthma severity score, using physiologic data. Using an ANN trained with three vital sign parameters, the pARS was well within 2 points of recorded PAS scores based on the analysis of MAE. This level of accuracy makes the automated score non-inferior to the manual PAS score. The pARS was most accurate in the mid-range of asthma severity between 6 and 9. This severity range is when critical decisions about patient care (transfer to ICU vs floor) are made, increasing the potential positive impact of an objective decision support tool that uses pARS.

The secondary electronic medical data merged with the passive vital sign monitoring information collected in the course of patient care created an automated pediatric respiratory score. The strengths of this machine score are objectivity, and that the machine score uses data collected automatically from monitors already in use. The machine score also incorporates age-based parameters and continuously monitored acute changes in asthma status with a computed score.

Because the machine score is objective, automated, and can be continuously generated, this score has the potential to help standardize acute pediatric asthma care in the PICU. Studies of PICU management of severe asthma across pediatric hospitals and even within a single institution have revealed marked variability in practice. While there are global treatment guidelines for asthma in the primary care and emergency department setting, acute care of life-threatening pediatric asthma, particularly in severe exacerbations treated in PICU, is not standardized. Respiratory scoring systems for pediatric asthma have streamlined and improved inpatient care, but there is an inconsistent use of any such scoring system in the PICU. The implemented machine learning engine resulted in a decreased length of stay in the hospital overall. An automated, quantitative score to replace these respiratory scores may be appealing to critical care providers who are concerned about evidence-based clinical decision making, interrater reliability, and staff efficiency.

Currently, no automated asthma severity score has been published that is sensitive to changes in acute respiratory status. Existing work in pediatric asthma prediction has focused on predicting the occurrence of asthma exacerbations and asthma control deterioration. Other machine learning and predictive analytic work in pediatrics have targeted sentinel events such as sepsis, respiratory failure requiring intubation, and cardiac arrest as outcomes. The frequency of these events is low and thus requires a large patient population and significant monitoring time to assess validity. The pARS score can be validated over a shorter time frame and in smaller populations because it assesses and learns the continuum of disease severity and can be associated with more common outcomes, for example, decreasing length of stay in PICU rather than leveraging outcomes such as respiratory arrest and death that are discrete rare events.

Limitations of this study include incomplete data from the clinical record and vital sign database, which decreased the inclusion cohort. While data fidelity issues are innate to using electronic clinical record data for secondary research investigation, this highlights the importance of considering issues of data input and integrity as health data systems are constructed, particularly as it becomes imperative to merge and map multiple data sources. Because of the practice patterns in the PICU, the PAS score is not recorded as frequently as on the inpatient floors, which limited data analysis, further emphasizing the need for an automated score. In addition, the machine learning engine training was restricted to the specific PAS score used at the center and therefore may not generalize to systems that use other versions of manual clinical scoring. The score was able to be built using a heterogenous pediatric patient population at altitude (˜5280 ft) seen at an urban quaternary care center. All patients included were on oxygen while in the PICU as a part of the standard of care. This introduces variability into the pulse oximetry data incorporated into the machine learning engine. Future prospective, multicenter study is needed to further elucidate the impact of oxygen therapy on vital sign parameters.

The machine learning engine may not include other clinical features or risk factors that can influence asthma severity. For example, acute bronchodilator use, the effect of noninvasive ventilation, as well as markers of chronic asthma severity including controller medication use and adherence, atopy, prior hospitalization history, recent exacerbations, symptom surveys, baseline FEV1%, and environmental exposures could all impact pARS predictive ability. Prospective collection of some of these parameters in future studies may strengthen the model and allow further personal risk stratification and treatment algorithms.

The study analyzed vital sign parameters measured at 1-minute intervals. The ability to apply deep or unsupervised machine learning to high data density vital sign waveforms has enormous predictive potential. For example, trends in heart rate variability have been used to predict neonatal sepsis and arterial waveforms analyzed to predict hypovolemic shock. Advances in mobile and hospital-based monitoring and cloud-based analytics are innovations that will help facilitate research and clinical application of high-resolution physiologic data, conquering current challenges of poor fidelity data due to artifact and variable collection, and the size of the data files.

Any decision support tool is only useful if providers trust and utilize it. Implementation and adoption of this tool in the PICU will require significant changes to workflow with patients and the use of the EHR. Significant quality improvement and culture change work will be needed to adopt this scoring system as a clinical decision-making tool. Given these limitations and context, the next steps may include work to prospectively validate pARS as a research instrument and associate it with meaningful clinical outcomes. Technologic innovation addressing issues including integration of data sources and the EHR, computing power, and the speed to run machine learning methods continuously may be required to translate this score into a clinician decision support tool. With prospective validation, real-time implementation and workflow adoption, a score like pARS can drive higher quality care, improve patient flow, decrease the length of stay and medication burden (facilitate timely weaning of continuous medications).

This study shows that the creation of a pARS leveraging machine learning techniques such as ANNs to analyze simple vital sign parameters and limited clinical data can be feasible. The potential impact of such a score to improve and standardize the PICU management of acute asthma exacerbation can be significant. The study revealed multiple barriers to integration of disparate clinical data sources and was also weakened by incomplete EHR and monitor data, both common challenges in studies using secondary retrospective queries on data produced by routine clinical practice. Further prospective validation of the machine learning engine can be imperative to improve data integrity, refine and expand contributing features, and assess the impact of pARS on clinical outcomes including length of stay, and medication burden and operational impacts such as staff efficiency.

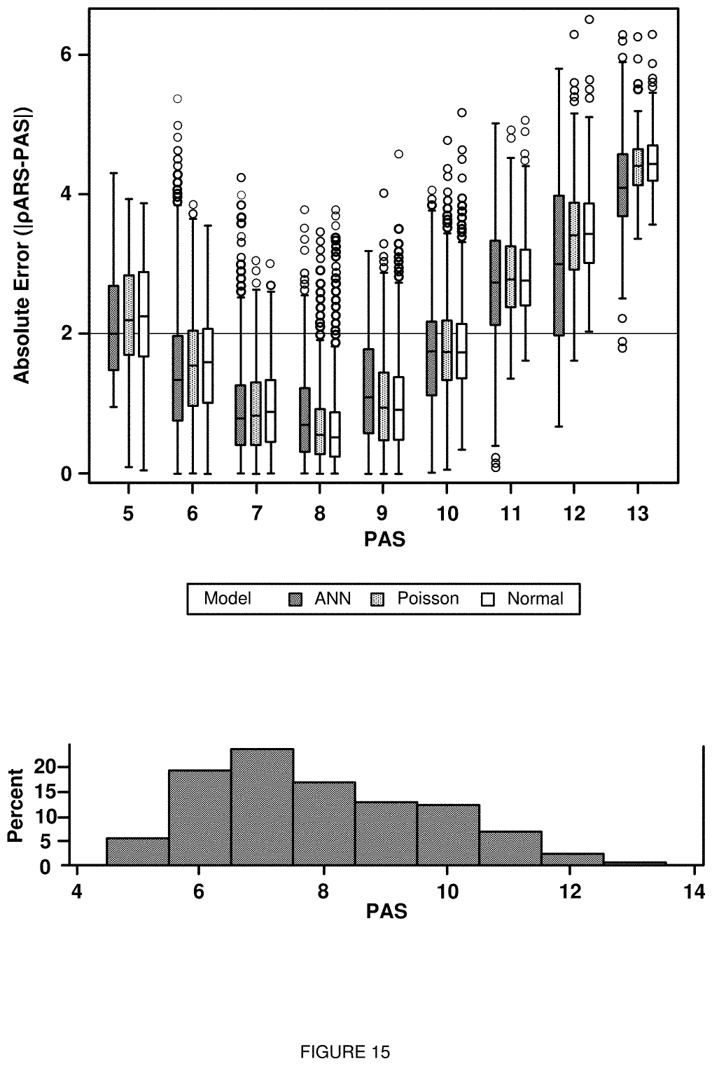

FIG. 15 is a plot illustrating the absolute error of a pediatric automated asthma respiratory score (pARS) plotted at each manual pediatric asthma score (PAS) value for an artificial neural network (ANN) implemented by a machine learning engine according to some embodiments of the present technology. Poisson and Normal models are also plotted in the balanced test set. In some embodiments, continuous vital sign monitoring data (heart rate, respiratory rate, and pulse oximetry) can be merged with health record data of a patient to determine the current state of a medical condition of a patient. In other embodiments, a cascaded artificial neural network (ANN) can be applied to create an automated respiratory score and validated by two approaches. FIG. 15 shows the ANN compared with the Normal and Poisson regression models. The distribution of the absolute errors are represented using box plots, the boxes extend to the 25th and 75th percentiles, the median values are indicated with a line inside the box and means are denoted with a large circle. The whiskers extend to 1.5 times the interquartile range and values outside of that are indicated with points. A reference line at 2 is plotted. A histogram of the PAS scores is displayed at the bottom of the FIG. 15.

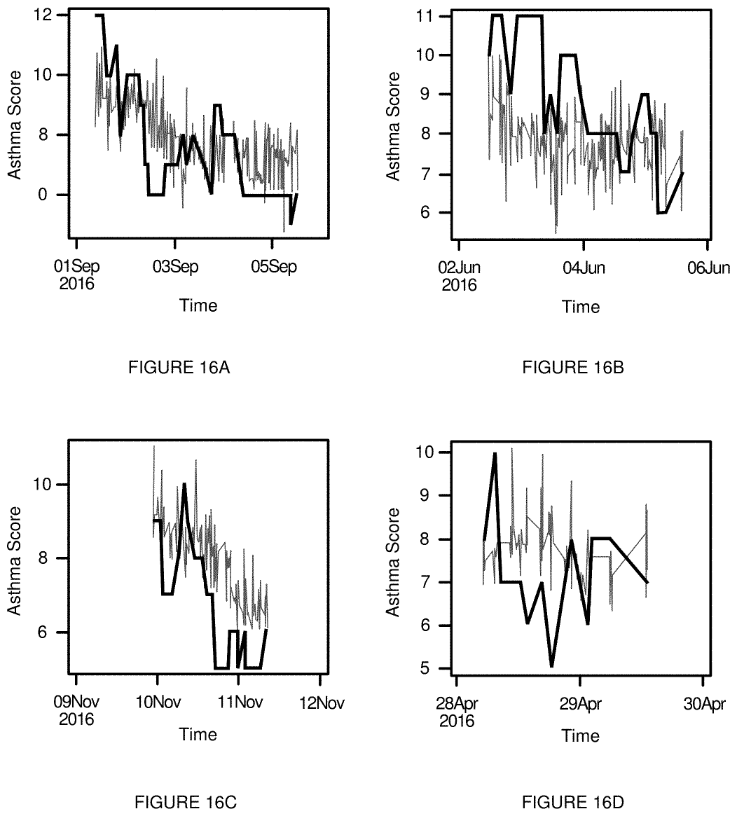

FIGS. 16A-16D are plots comparing the pARS scores (gray lines) overlaid on PAS scores (thick black lines) plotted over time for four example subjects according to one or more embodiments of the present technology. FIGS. 16A-16D each correspond to a single patient. FIGS. 16A-16D demonstrate a loose correlation between the pARS scores and the PAS scores.

Referring again to FIG. 17A, care guideline(s) may be stored in at least one memory device 270. In the illustrated embodiment, memory device 270 storing care guidelines may be positioned remote from (e.g., in the cloud), and in communication with, mobile device 240. In other embodiments, memory device 270 may be positioned in the integrated unit 255 and/or may be part of memory device(s) of the mobile device 240 itself. Memory device 270 may be in wired and/or wireless communication with the above described machine learning (ML) engine (e.g., ML engine 109, which may also be referred to herein as artificial intelligence (AI) engine). ML engine 109 may include associated memory and processor(s), not shown in FIG. 17A, facilitating the operations and processes described above. In the illustrated embodiment, ML engine 109 may be positioned remote from (e.g., in the cloud), and in communication with, mobile device 240. In other embodiments, ML engine 109 may be positioned in the integrated unit 255 and/or may be part of processor(s) and/or memory device(s) of the mobile device 240 itself.

In an example embodiment, care guidelines stored in memory device 270 receives biosignals 265 from the IPC device 245 measured by each sensing module 210, as shown in FIG. 17A. Application(s) running on mobile device 240 may perform preprocessing operations such as decoding and encrypting prior to or concurrent with data communication components of mobile device 240 transmitting these data to the care guidelines stored in memory device 270. In embodiments of the disclosed systems and methods that either employ or do not utilize the ML engine 109, the biosignals 265 may be required to be frequently inputted to the care guidelines stored in memory device 270. Therefore, the availability of a portable sensing platform such as the combination of IPC device 245 and modular sensing module(s) 210 in the unit 255 shown in FIG. 17A may facilitate completion of above-described functions of the care guidelines for clinicians and their patients, among other users. The care guideline(s) may be inaccurate if the received data contained in the biosignals 265 is infrequent. Known devices, systems and methods that may be related to the embodiments disclosed herein may be deficient in providing this function because of their lack of coordination between multiple devices and options to select the preferred module to make it convenient for the users.

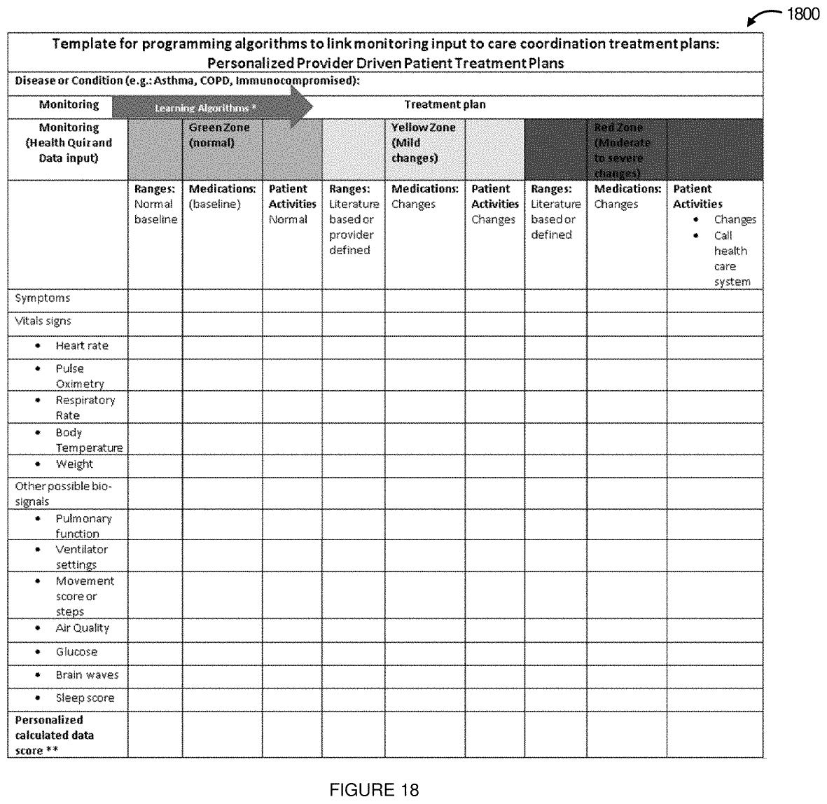

FIG. 18 is a data structure 1800 providing a template for programming algorithms to link monitoring input to care coordination treatment plans to facilitate personalized provider driven patent treatment plans, according to some embodiments of the present technology. The data structure 1800 may facilitate development and updating of the care guidelines stored in memory device 270, as described above with reference to FIG. 17A. Referring to the left-most column in the table representing data structure 1800 in FIG. 18, “vital signs” and “other possible biosignals” may be received from, for instance, unit 255 via the mobile device 240. Further data for use by algorithms (with or without ML or AI) data structure 1800 may include “symptoms” input by, for example, a patient using the disclosed health sticker and/or other devices capable of monitoring vital signs and other data. Upon receipt of these data, and with subsequent updating of these data on a continuous or periodic basis, algorithms associated with data structure 1800 process the data to then formulate and/or update a “treatment plan,” as shown in FIG. 18. Software with associated GUIs such as NOW VITAL (www.f6s.com/nowvitals, Fort Collins, Colo.) may mediate or otherwise facilitate this processing to, for example, provide timely alerts of changes in “monitoring” status to patients and providers. Accordingly, changes in biosignal(s) 265 may be accurately tracked, quantified, and stored, and thereby used to automatically change treatment plan(s) and provide clinicians timely opportunity to review such updates and intervene with patients and/or other provides quickly, as needed.

In the case of utilization of ML or AI with the disclosed systems and methods according to the present technology, learning algorithms implemented by processor(s) of, for example, ML engine 109 may take the input from the monitored signals—symptoms, vital signs or other biosignals—and tracks it to the provider programmed treatment plans. This capability, among others, of the present technology provides substantially more than mere remote monitoring or treatment plans by specifically personalizing them to particular patients. Such computing techniques and tangible technical benefits and practical applications thereof to providers and patients are not provided by known systems and methods, and, as shown in the bottom-most row of the data structure 1800 table of FIG. 18, further enable providing personalized data scores to be calculated and updated at or near real-time (e.g., within a time limited only by delays caused by data transmission and by the computational speeds of processors used for the computations). Such scores may be computed in manner that provides a convenient way for patients and provides to quickly take meaningful actions to intervene and, as such, may improve both patient outcomes in either preventative or interventional contexts, and the efficiency and effectiveness of patient care.

### Exemplary Computer System Overview

Aspects and implementations of the systems and methods of the present disclosure have been described in the general context of various steps and operations. A variety of these steps and operations may be performed by hardware components or may be embodied in computer-executable instructions, which may be used to cause a general-purpose or special-purpose processor (e.g., in a computer, server, or other computing device) programmed with the instructions to perform the steps or operations. For example, the steps or operations may be performed by a combination of hardware, software, and/or firmware.

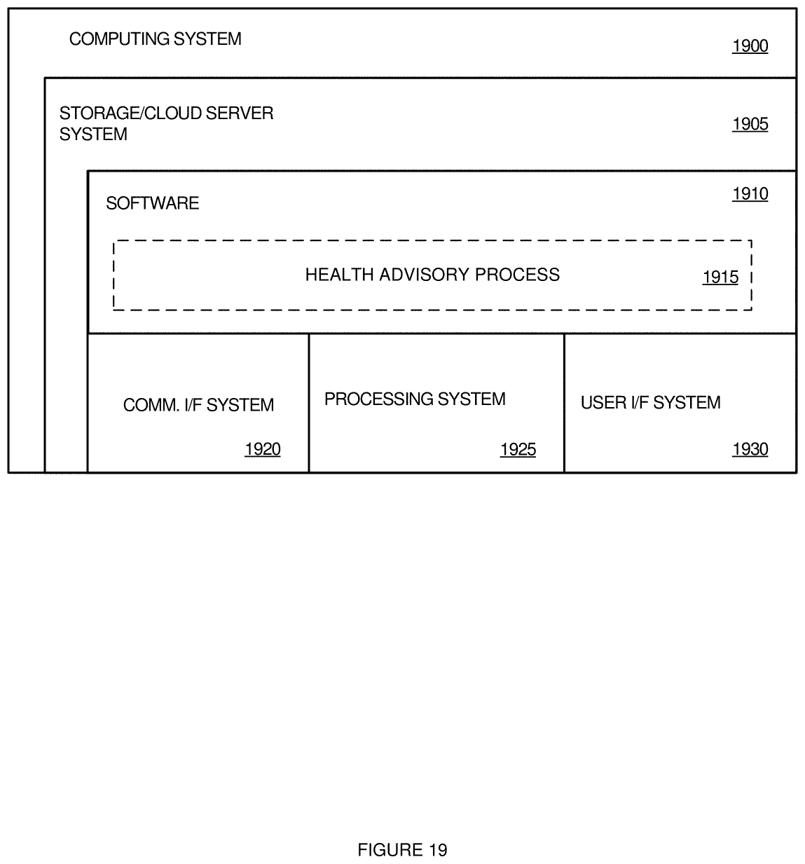

FIG. 19 illustrates computing system 1900 that is representative of any system or collection of systems in which the various processes, programs, services, and scenarios disclosed herein may be implemented. Examples of computing system 1900 include, but are not limited to, server computers, routers, web servers, cloud computing platforms, and data center equipment, as well as any other type of physical or virtual server machine, physical or virtual router, container, and any variation or combination thereof.

Computing system 1900 may be implemented as a single apparatus, system, or device or may be implemented in a distributed manner as multiple apparatuses, systems, or devices. Computing system 1900 includes, but is not limited to, processing system 1925, storage system 1905, software 1910, communication interface system 1920, and user interface system 1930 (optional). Processing system 1925 is operatively coupled with storage system 1905, communication interface system 1920, and user interface system 1930.

Processing system 1925 loads and executes software 1910 from storage system 1905. Software 1910 includes and implements health advisory process 1915, which is representative of the health advisory process as discussed with respect to the preceding Figures. When executed by processing system 1925, software 1910 directs processing system 1925 to operate as described herein for at least the various processes, operational scenarios, and sequences discussed in the foregoing implementations. Computing system 1900 may optionally include additional devices, features, or functionality not discussed here for purposes of brevity.

Processing system 1925 may comprise a micro-processor and other circuitry that retrieves and executes software 1910 from storage system 1905. Processing system 1925 may be implemented within a single processing device but may also be distributed across multiple processing devices or sub-systems that cooperate in executing program instructions. Examples of processing system 1925 include general purpose central processing units, graphical processing units, application specific processors, and logic devices, as well as any other type of processing device, combinations, or variations thereof.

Storage system 1905 may comprise any computer readable storage media that is readable by processing system 1925 and capable of storing software 1910. Storage system 1905 may include volatile and nonvolatile, removable and non-removable media implemented in any method or technology for storage of information, such as computer readable instructions, data structures, program modules, or other data. Examples of storage media include random access memory, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 1905 may also include computer readable communication media over which at least some of software 1910 may be communicated internally or externally. Storage system 1905 may be implemented as a single storage device but may also be implemented across multiple storage devices or sub-systems co-located or distributed relative to each other. Storage system 1905 may comprise additional elements, such as a controller, capable of communicating with processing system 1925 or possibly other systems.

Software 1910 (health advisory process 1915) may be implemented in program instructions and among other functions may, when executed by processing system 1925, direct processing system 1925 to operate as described with respect to the various operational scenarios, sequences, and processes illustrated herein. For example, software 1910 may include program instructions for implementing a health advisory process as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 1910 may include additional processes, programs, or components, such as operating system software, virtualization software, or other application software. Software 1910 may also comprise firmware or some other form of machine-readable processing instructions executable by processing system 1925.

In general, software 1910 may, when loaded into processing system 1925 and executed, transform a suitable apparatus, system, or device (of which computing system 1900 is representative) overall from a general-purpose computing system into a special-purpose computing system customized to optimize secure traffic as described herein. Indeed, encoding software 1910 on storage system 1905 may transform the physical structure of storage system 1905. The specific transformation of the physical structure may depend on various factors in different implementations of this description. Examples of such factors may include, but are not limited to, the technology used to implement the storage media of storage system 1905 and whether the computer-storage media are characterized as primary or secondary storage, as well as other factors.

For example, if the computer readable storage media are implemented as semiconductor-based memory, software 1910 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 1920 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 1900 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology may be recited in a particular claim format (e.g., method claim, system claim, computer-readable medium claim), other aspects may likewise be embodied in these or other claim formats, such as being embodied in a means-plus-function claim. Any claims intended to be treated under 35 U.S.C. § 112(f) will begin with the words “means for”, but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

What is claimed is:

1. A system to facilitate medical diagnostics via interchangeable medical devices, the modular platform comprising:
an adhesive side having an adhesive layer to affix a medical device platform to a mobile device;
a device side having a device port configured to receive a medical device to monitor a vital sign of a user;
a data module to create medical information from the vital sign collected by the medical device;
a communication module to transmit the medical information to the mobile device; and
a power management module to distribute power to the one or more medical devices, the data module, and the communication module.

2. The system of claim 1, wherein the mobile device generates graphical user interface (GUI) comprising one or more modes of operation:
a first mode of operations that visually presents the medical information as one or more visual elements or textual elements which are updated in real time in response to changes in the medical information;
a second mode of operation configured to generate one or more visual notifications or audio notifications in response to the changes in the medical information; and
a third mode of operation to present an interactive questionnaire relating to a medical condition of the user to assist in identifying a severity level of the medical condition of the user.

3. The system of claim 1, wherein the device side has multiple ports and the communication module sends an indication of which medical devices are available via the multiple ports and generates a graphical user interface (GUI) allowing the user to select which of the medical devices to use.

4. The system of claim 1, further comprising a machine learning engine configured to ingest the medical information and determine a severity level of the medical information indicative of a current medical condition of the user.

5. The system of claim 1, wherein the one or more device ports further comprise:
a slide and lock mechanism to hold in place the medical device when the medical device is inserted into the device port; and
a device adapter that connects the one or more medical devices to the data module.

6. The system of claim 1, wherein the power management module detects when the medical device is inserted or removed into the device port and automatically directs generates an alert indicating the medical device is available or unavailable for use.

7. The system of claim 1, wherein the data module develops correlations between vital signs originating from multiple different medical devices to determine medical information that is not directly collected by any of the multiple medical devices.

8. A method to facilitate medical diagnostics, the method comprising:
measuring, via a modular medical device platform, one or more vital signals of a user;
correlating, via a machine learning engine, the vital signals of the user to a medical condition of the user to develop a health score indicative of a current state of the medical condition of the user;
constructing, based on the health score, an action plan comprising a prescribed response to the current state of the medical condition of the user and once the action plan is constructed;
presenting the action plan to the user; and
requesting confirmation from the user that the user has followed the action plan.

9. The method of claim 8, further comprising presenting an interactive health questionnaire to the user that includes questions relating to the current state of the medical condition of the user and upon completion of the interactive health questionnaire by the user, updating the current state of the medical condition of the user.

10. The method of claim 8, further comprising generating, in real time, a visual representation of the vital signs of the user.

11. The method of claim 8, further comprising generating an alert indicative of a change in the current state of the medical condition of the user, wherein generating the alert includes sending one or more of an audio alert or a visual alert.

12. The method of claim 8, wherein the modular medical device platform comprises an adhesive layer that secures the medical device platform to a cell phone and one or more device ports to hold one or more medical devices to monitor one or more vital signs of a user and collect the one or more vital signs of the user as health metrics.

13. The method of claim 8, wherein correlating the vital signals of the user to the medical condition of the user includes determining, via the machine learning engine, a current state of the medical condition of the user and, notifying the user of the current state of the medical condition.

14. A cloud-based data aggregation engine to facilitate medical diagnostics, the could-based data aggregation engine comprising:
a communication module to receive medical information from mobile devices,
wherein each of the mobile devices are connected to a medical device platform having one or more ports supporting interchangeable medical devices to measure vital signs of a user and develop medical information of the user based on the vital signs;
wherein the mobile devices report, to the cloud-based data aggregation engine, the medical information of the user associated with the mobile devices;
a machine learning engine to ingest the medical information and process the medical information to determine a health score of each user that is indicative of a current state of a medical condition of each user; and
a health assessment module to distribute health questionnaires each relating to the medical condition of each user to the mobile devices, and upon completion of the questionnaire by each user, update the health score of each user.

15. The cloud-based data aggregation engine of claim 14, wherein the health assessment module dynamically generates action plans that include a prescribed response to the current state of the medical condition of each user based on the updated health score of each user, and distributes the action plans to the mobile devices.

16. The cloud-based data aggregation engine of claim 15, wherein the action plans further include levels that indicate a severity of the current state of the medical condition of each user, and wherein the updated health score dictates the level.

17. The cloud-based data aggregation engine of claim 14, wherein the communication module reports the medical information of each user and the current state of the medical condition of each user to an external health resource.

18. The cloud-based data aggregation engine of claim 14, wherein the machine learning engine generates predictions to determine a future state of the medical condition of each user based on the medical information.

19. The cloud-based data aggregation engine of claim 14, wherein the one or more ports include:
a slide and lock mechanism to hold in place the medical device when the medical device is inserted into the device port; and
a device adapter that connects the one or more medical devices to a data module.

20. The cloud-based data aggregation engine of claim 14, wherein the interchangeable medical devices include invasive medical devices and non-invasive medical devices.

21. The cloud-based data aggregation engine of claim 14, further comprising a feedback loop to remind users to take medication and receive confirmation of medication adherence.

22. The cloud-based data aggregation engine of claim 14, wherein the communication module receives environmental data from an external source and the machine learning engine ingests the environmental data as part of determining the health score of each.
